(19) United States
(12) Patent Application Publication
(10) Pub. No.: US 2026/0139320 A1
(43) Pub. Date: May 21, 2026

(54) StarTrace: A Multiplex Drug Testing Platform for Personalized Medicine

(71) Applicant: UNIVERSITY OF SOUTH CAROLINA, COLUMBIA, SC (US)

(72) Inventors: SANA KHALILI, LEXINGTON, SC (US); PHILLIP BUCKHAULTS, BLYTHEWOOD, SC (US); SHREY PATEL, COLUMBIA, SC (US); CAROLYN BANISTER, COLUMBIA, SC (US)

(21) Appl. No.: 19/345,270

(22) Filed: Sep. 30, 2025

Related U.S. Application Data
(60) Provisional application No. 63/721,702, filed on Nov. 18, 2024.

(51) Int. Cl. C12Q 1/6886; G01N 33/50

(52) U.S. Cl. CPC C12Q 1/6886; G01N 33/5011; G01N 33/5082; C12Q 2600/136; C12Q 2600/156

(57) ABSTRACT

In general, disclosed herein are methods for simultaneous monitoring the relative sensitivities of different organoids to a therapeutic agent. The method may include obtaining a sample that includes a plurality of barcoded organoid avatars; contacting the sample with a therapy; detecting in the sample at a first point in time the copy number, amount, and/or activity of one or more organoids; repeating the detection step at a subsequent point in time; and comparing the copy number of one or more organoids detected to monitor the progression of the effectiveness of the therapy.

### CROSS REFERENCE TO RELATED APPLICATIONS

This application claims the filing benefit of U.S. Provisional Patent Application Ser. No. 63/721,702, filed on Nov. 18, 2024, which is incorporated herein by reference.

### SEQUENCE LISTING

The instant application contains a Sequence Listing which has been submitted electronically in XML format and is hereby incorporated by reference in its entirety. Said XML copy, created on Dec. 16, 2025, is named USC-843_1744_SL.xml and is 88,248 bytes in size.

### BACKGROUND

The development of cancer treatments tailored to the needs of individual patients is a promising paradigm for oncology. Increased utilization of tumor sequencing illustrates the growing interest in using therapies that specifically target genetic variations in tumors. However, while genetic sequencing has advanced the identification of targetable mutations, only a small percentage of tumors have mutations known to be actionable, and even those that do may not always respond predictably to treatment. Many tumors, despite lacking clear genetic indicators, may still be highly sensitive to drugs that are not typically considered for those patients. The disconnect between somatic mutations and therapeutic response highlights the need for more versatile approaches to cancer treatment.

In vitro anticancer drug screens have traditionally been performed using 2D cancer cell lines, often using concentrations of drug that produce cell death within 2-3 days. However, these preclinical results often correlate poorly with animal models or with actual clinical outcomes. There is an unmet need for more relevant preclinical models that can be used at the early stages of drug discovery, such as high-throughput screening (HTS). Preclinical models should represent the recurring genetic themes present in real human cancers, enabling more relevant preclinical results and product development decisions. Patient-derived organoid avatars (PDOs) are sophisticated and highly relevant in vitro models. Organoids are self-organizing mammalian adult stem cells and are strong tools for ex vivo tissue morphogenesis and organogenesis simulations. Because cancer and normal organoids contain the array of germline and somatic mutations that influence drug response, this technology has the potential to bridge the gap between our basic science understanding of cancer genetics and pragmatic testing of new treatments for patients. By using PDOs, it is possible to capture the heterogeneity of human tumors, providing a more accurate readout of potential treatment efficacy. Organoids can be used alongside traditional methods such as cell-line and xenograft-based drug research and can enable individualized therapy design.

As such, a need exists in the arts for methods for simultaneous monitoring the relative sensitivities of different organoids to a therapeutic agent.

### SUMMARY

In general, disclosed herein are methods for simultaneous monitoring the relative sensitivities of different organoids to a therapeutic agent. The method may include obtaining a sample that includes a plurality of barcoded organoid avatars; contacting the sample with a therapy; detecting in the sample at a first point in time the copy number, amount, and/or activity of one or more organoids; repeating the detection step at a subsequent point in time; and comparing the copy number of one or more organoids detected to monitor the progression of the effectiveness of the therapy.

Other features and aspects of the present disclosure are discussed in greater detail below.

### BRIEF DESCRIPTION OF THE DRAWINGS

A full and enabling disclosure of the present disclosure is set forth more particularly in the remainder of the specification, including reference to the accompanying figures, in which:

FIG. 1 illustrates the workflow of StarTrace, which includes isolation of individual DNA barcodes from the ClonTracer barcode virus library and workflow of barcoding and tracking PDOs within a mixture by both NGS and StarTrace PCR.

FIG. 2A depicts a heatmap showing the pairwise hamming distances between 12 barcodes in the BC Pool-1. Each cell represents the hamming distance between two barcodes.

FIG. 2B depicts a heatmap showing the pairwise hamming distances between 15 barcodes in the BC Pool-2. Each cell represents the hamming distance between two barcodes.

FIG. 3A depicts sand plots showing the percentage survival of each barcoded organoids within the pool-1 over 28 days of treatment with drugs, only DMSO, Nutlin, Mercaptopurine, and Ibrutinib are shown. Each line represents an organoids's survival trajectory, demonstrating how its proportion relative to the total pool changes over time.

FIG. 3B depicts sand plots showing the percentage survival of each barcoded organoids within the pool-2 over 27 days of treatment with Nutlin, LGK-974, and Etoposide. Each line represents an organoids's survival trajectory, demonstrating how its proportion relative to the total pool changes over time.

FIG. 4A depicts nutlin of BC pool-1 PDOs over time, deconvoluted by NGS. Log-fold change in frequency of each barcoded PDOs within the pool-1, treated with nutlin for 28 days. Each violin represents the log-fold change in a specific barcode relative to vehicle control.

FIG. 4B depicts ibrutinib of BC pool-1 PDOs over time, deconvoluted by NGS. Log-fold change in frequency of each barcoded PDOs within the pool-1, treated with ibrutinib for 28 days. Each violin represents the log-fold change in a specific barcode relative to vehicle control.

FIG. 4C depicts lapatinib of BC pool-1 PDOs over time, deconvoluted by NGS. Log-fold change in frequency of each barcoded PDOs within the pool-1, treated with lapatinib for 28 days. Each violin represents the log-fold change in a specific barcode relative to vehicle control.

FIG. 5A depicts log fold change of both ERBB3 mutant frequency and barcoded F173T PDO frequency over time in presence of Ibrutinib. PDO frequency was determined by sequencing both the barcode amplicon and an ERBB3 amplicon flanking the Val104Leu mutant allele.

FIG. 5B depicts ibrutinib dose-response of F173T tumor and its matched normal F773N. The ERBB3 mutant allele frequency in a mixture of F173T (tumor) and F173N (normal) PDOs treated with Ibrutinib. PDO frequency was determined by sequencing an ERBB3 amplicon flanking the Val104Leu mutant allele. Data points represent the average of ERBB3 mutant allele frequency from three biological replicates.

FIG. 6A depicts nutlin responses of BC pool-2 PDOs over time, deconvoluted by NGS. Log-fold change in frequency of each barcoded PDOs within the pool-2, treated with nutlin for 27 days. Each violin represents the log-fold change in a specific barcode relative to vehicle control.

FIG. 6B depicts LGK974 responses of BC pool-2 PDOs over time, deconvoluted by NGS. Log-fold change in frequency of each barcoded PDOs within the pool-2, treated with LGK974 for 27 days. Each violin represents the log-fold change in a specific barcode relative to vehicle control.

FIG. 6C depicts etoposide responses of BC pool-2 PDOs over time, deconvoluted by NGS. Log-fold change in frequency of each barcoded PDOs within the pool-2, treated with etoposide for 27 days. Each violin represents the log-fold change in a specific barcode relative to vehicle control.

FIG. 7A depicts dose response curve of LGK974 on a mixture of two BC PDOs, deconvoluted by StarTrace PCR. F147T_P1-G6 carried an RNF43 mutation and is sensitive to PORCN inhibitors (LGK-974 and wnt-C59). The ratios of the two tumors were measured by StarTrace PCR and plotted against the log concentration of LGK-974 and Wnt-C59.

FIG. 7B depicts Dose response curve of Wnt-C59 on a mixture of two BC PDOs, deconvoluted by StarTrace PCR. F147T_P1-G6 carried an RNF43 mutation and is sensitive to PORCN inhibitors (LGK-974 and wnt-C59). The ratios of the two tumors were measured by StarTrace PCR and plotted against the log concentration of LGK-974 and Wnt-C59.

FIG. 8A depicts nutlin responses of BC pool-2 PDOs over time, deconvoluted by StarTrace PCR. Log-fold change in frequency of each barcoded PDOs within the pool-2, treated with nutlin for 10 days. Each violin represents the log-fold change in in a specific barcode relative to vehicle control.

FIG. 8B depicts LGK974 responses of BC pool-2 PDOs over time, deconvoluted by StarTrace PCR. Log-fold change in frequency of each barcoded PDOs within the pool-2, treated with LGK974 for 10 days. Each violin represents the log-fold change in in a specific barcode relative to vehicle control.

FIG. 8C depicts etoposide responses of BC pool-2 PDOs over time, deconvoluted by StarTrace PCR. Log-fold change in frequency of each barcoded PDOs within the pool-2, treated with etoposide for 10 days. Each violin represents the log-fold change in in a specific barcode relative to vehicle control.

FIG. 9A depicts comparison of the log-fold change in barcoded PDOs frequency obtained by NGS and StarTrace PCR for Nutlin.

FIG. 9B depicts comparison of the log-fold change in barcoded PDOs frequency obtained by NGS and StarTrace PCR for LGK-974.

FIG. 9C depicts comparison of the log-fold change in barcoded PDOs frequency obtained by NGS and StarTrace PCR for Etoposide.

FIG. 10 depicts StarTrace PCR on the Pool-2 miniaturize assay. Clustered heatmap of the log fold changes in PDO frequencies over 10 days in presence of different drugs.

FIG. 11 depicts Sanger sequencing results and assembly of each 72 unique DNA barcode in relation to each other (SEQ ID NOs 20-29, respectively, in order of appearance).

FIG. 12A depicts 5-FU response of BC pool-1 PDOs over time, deconvoluted by NGS. Log-fold change in frequency of each barcoded PDOs within the pool-1, treated with 5-FU for 28 days. Each violin represents the log-fold change in a specific barcode relative to vehicle control.

FIG. 12B depicts mercaptopurine response of BC pool-1 PDOs over time, deconvoluted by NGS. Log-fold change in frequency of each barcoded PDOs within the pool-1, treated with mercaptopurine for 28 days. Each violin represents the log-fold change in a specific barcode relative to vehicle control.

Repeat use of reference characters in the present specification and drawings is intended to represent the same or analogous features or elements of the present invention.

### DETAILED DESCRIPTION

Reference will now be made in detail to various embodiments of the presently disclosed subject matter, one or more examples of which are set forth below. Each embodiment is provided by way of explanation, not limitation, of the subject matter. In fact, it will be apparent to those skilled in the art that various modifications and variations may be made to the present disclosure without departing from the scope or spirit of the disclosure. For instance, features illustrated or described as part of one embodiment may be used in another embodiment to yield a still further embodiment. Thus, it is intended that the present disclosure covers such modifications and variations as come within the scope of the appended claims and their equivalents.

In general, disclosed herein are methods for simultaneous monitoring the relative sensitivities of different organoids to a therapeutic agent. The method may include obtaining a sample that includes a plurality of barcoded organoid avatars; contacting the sample with a therapy; detecting in the sample at a first point in time the copy number, amount, and/or activity of one or more organoids; repeating the detection step at a subsequent point in time; and comparing the copy number of one or more organoids detected to monitor the progression of the effectiveness of the therapy.

As used herein, an “organoid avatar” refers to self-organized three-dimensional tissue cultures that are derived from stem cells or progenitor cells, and which recapitulate at least some of the structural, functional, and/or cellular characteristics of an organ or tissue of origin. In one example embodiment, an organoid avatar disclosed herein may include normal (e.g., non-inflammatory) organoids or disease organoids. In one example embodiment, an organoid avatar disclosed herein may be a patient-derived organoid (PDO) avatar, which refers to organoids that may be established from primary cells, tissues, or biopsy specimens obtained from a human.

In one example embodiment, organoids disclosed herein may be derived from epithelial cells. For instance, organoids disclosed herein may be derived from epithelial cells including, but not limited to, colorectal cells, intestinal cells, crypt cells, rectal cells, lung cells, liver cells, breast cells, skin cells, pancreatic cells, endocrine cells, exocrine cells, ductal cells, renal cells, adrenal cells, thyroid cells, pituitary cells, parathyroid cells, prostate cells, stomach cells, oesophageal cells, ovary cells, or a combination thereof. In one example embodiment, organoids may be colorectal organoids. In another example embodiment, organoids may be liver organoids. In another example embodiment, organoids may be breast organoids.

In one example embodiment, organoids may be obtained from a biological sample. For instance, in one example embodiment, a biological sample may include, but is not limited to, a tissue sample (e.g., biopsy) or a surgical resection tissue. Colorectal organoid avatars may be obtained, for instance, from a resected colon and/or rectum for colorectal epithelial cells.

Organoids utilized herein may be tagged with a barcode to generate a barcoded organoid avatar. In some example embodiments, organoid avatars may include a barcode attached to an organoid. As used herein, a “barcode” refers to a unique sequence of nucleotides that allows identification of the nucleic acid of which the barcode is a part. Barcoding organoids is a process by which the organoid may be uniquely tagged with one or more short identifying sequences. In one example embodiment, each organoid in a sample may have a barcode that is unique from barcodes on any other organoid in the sample. For instance, in one example embodiment, each organoid in a sample may have one unique barcode. In another example embodiment, each organoid in a sample may include more than one barcode that are unique from each other and unique from any other barcode that may be attached to any other organoid the sample.

In one example embodiment, the barcode may be attached directly to an organoid avatar disclosed herein. For instance, the barcode may be covalently or noncovalently attached to an organoid avatar disclosed herein. In another example embodiment, the barcode may be attached indirectly to an organoid avatar disclosed herein.

In one example embodiment, organoid avatars may include a nucleic acid barcode. Barcoded organoid avatars may include nucleotide sequences that provide a unique identifier for each organoid that may be utilized to detect, quantify, and/or measure organoids presence in a sample. In one example embodiment, the nucleic acid barcode may have a sequence length of from about 6 base pairs (bp) to about 50 bp, such as from about 8 bp to about 34 bp, such as from about 12 bp to about 30 bp, or any range therebetween. For instance, in one example embodiment, the nucleic acid barcode may have a sequence length of from about 8 bp to about 34 bp. In another example embodiment, the nucleic acid barcode may have a sequence length of from about 12 bp to about 30 bp.

In one example embodiment, the nucleic acid barcode may have a sequence of 20 bp. In another example embodiment, the nucleic acid barcode may have a sequence of 30 bp. In another example embodiment, the nucleic acid barcode may have a sequence of 34 bp. In another example embodiment, the nucleic acid barcode may have a sequence of 50 bp.

A barcoded organoid avatar described herein may be produced using standard recombinant techniques. For instance, techniques may include the use of vectors, such as expression vectors, containing a nucleic acid barcode. As used herein, “vector” refers to a nucleic acid molecule capable of transporting another nucleic acid to which it has been linked. In one example embodiment, the vector may be a plasmid, which refers to a circular double stranded DNA loop into which additional DNA segments can be ligated. In another example embodiment, the vector may be a viral vector. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors, namely expression vectors, are capable of directing the expression of genes to which they are operably linked. In general, expression vectors of utility in recombinant DNA techniques are often in the form of plasmids (vectors). However, the present invention is intended to include such other forms of expression vectors, such as viral vectors (e.g., replication defective retroviruses), which serve equivalent functions.

In one example embodiment, the nucleic acid barcode may be produced in a viral vector. For instance, a viral vector may include, but is not limited to, lentiviral vectors, adenoviral vectors, adeno-associated viral (AAV) vectors, retroviral vectors, or a combination thereof. In one example embodiment, the nucleic acid barcode may be produced in a lentiviral vector. In another example embodiment, the nucleic acid barcode may be produced in adenoviral vectors. In another example embodiment, the nucleic acid barcode may be produced in adeno-associated viral (AAV) vectors. In another example embodiment, the nucleic acid barcode may be produced in retroviral vectors.

In one example embodiment, recombinant expression vectors may include a nucleic acid barcode disclosed herein in a form suitable for expression of the nucleic acid in a host cell. This means that the recombinant expression vectors include one or more regulatory sequences, selected on the basis of the host cells to be used for expression, which is operably linked to the nucleic acid sequence to be expressed. Within a recombinant expression vector, “operably linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” is intended to include promoters, enhancers and other expression control elements (e.g., polyadenylation signals). Regulatory sequences include those which direct constitutive expression of a nucleotide sequence in many types of host cell and those which direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, and the like. The expression vectors of the present invention can be introduced into host cells to thereby produce proteins or peptides, including fusion proteins or peptides, encoded by nucleic acids as described herein.

For instance, in one example embodiment, the nucleic acid barcode may be integrated into the genome of cell populations. In one example embodiment, the lentiviral vector may stably integrate into the genomes of the transduced cell populations. In another example embodiment, the retroviral vector may stably integrate into the genomes of the transduced cell populations.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid into a host cell, including calcium phosphate or calcium chloride co-precipitation, DEAE-dextran-mediated transfection, lipofection, or electroporation.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., for resistance to antibiotics) is generally introduced into the host cells along with the gene of interest. Preferred selectable markers include those which confer resistance to drugs, such as G418, hygromycin and methotrexate. Cells stably transfected with the introduced viral vector can be identified by drug selection (e.g., cells that have incorporated the selectable marker gene will survive, while the other cells die).

A nucleic acid barcode disclosed herein may be amplified using cDNA, mRNA, or genomic DNA as a template and appropriate oligonucleotide primers according to standard PCR amplification techniques. The nucleic acid molecules so amplified can be cloned into an appropriate vector and characterized by DNA sequence analysis. Furthermore, oligonucleotides corresponding to all or a portion of a nucleic acid barcode disclosed herein may be prepared by standard synthetic techniques, e.g., using an automated DNA synthesizer.

In one example embodiment, a barcoded nucleic acid may include a cohort of mixed population of organoids for screening and/or detecting. For instance, a barcoded nucleic acid cohort may include from about 50 or more unique nucleic acid barcodes for tagging an organoid. In one example embodiment, a barcoded nucleic acid cohort may include from about 60 or more unique nucleic acid barcodes for tagging an organoid. In another example embodiment, a barcoded nucleic acid cohort may include from about 70 or more unique nucleic acid barcodes for tagging an organoid. In another example embodiment, a barcoded nucleic acid cohort may include from about 80 or more unique nucleic acid barcodes for tagging an organoid. In another example embodiment, a barcoded nucleic acid cohort may include from about 90 or more unique nucleic acid barcodes for tagging an organoid. In another example embodiment, a barcoded nucleic acid cohort may include from about 150 or more unique nucleic acid barcodes for tagging an organoid.

In some example embodiments, two or more barcoded organoid avatars disclosed herein may be pooled to generate one or more barcoded polyclonal population of organoid avatars. A pool of barcoded organoid avatars may include from about 2 organoid avatars to about 1000 organoid avatars, such as from about 5 organoid avatars to about 950 organoid avatars, such as from about 10 organoid avatars to about 750 organoid avatars, such as from about 15 organoid avatars to about 500 organoid avatars, such as from about 20 organoid avatars to about 250 organoid avatars, or any range therebetween. In one example embodiment, a pool of barcoded organoid avatars may include from about 5 organoid avatars to about 950 organoid avatars. In another example embodiment, a pool of barcoded organoid avatars may include from about 10 organoid avatars to about 750 organoid avatars. In another example embodiment, a pool of barcoded organoid avatars may include from about 15 organoid avatars to about 500 organoid avatars. In another example embodiment, a pool of barcoded organoid avatars may include from about 20 organoid avatars to about 250 organoid avatars.

Methods disclosed herein are directed to screening a drug against one or more organoid avatars disclosed herein. For instance, methods may include monitoring a drug's ability to modulate (e.g., inhibit) a disease using one or more organoid avatars. In some example embodiments, methods for screening a drug may include culturing one or more organoid avatars disclosed herein, contacting the organoid avatars disclosed herein with the drug, and monitoring the drug effect in the organoid avatars disclosed herein.

In one example embodiment, one or more organoid avatars disclosed herein may be cultured in a culture media. For instance, the culture media may be a media that is suitable for organization and maintenance of one or more organoid avatars. In one example embodiment, the culture media may be selected from a group consisting of phosphate-buffered saline (PBS), 4-(2-Hydroxyethyl)piperazine-1-ethanesulfonic acid (HEPES), Dulbecco's Modified Eagle Medium (DMEM), Roswell Park Memorial Institute 1640 Medium, Ham's F-12 Medium (F12), and DMEM/F12. In one example embodiment, the culture media may be PBS. In another example embodiment, the culture media may be HEPES. In another example embodiment, the culture media may be DMEM. In another example embodiment, the culture media may be DMEM/F12.

In one example embodiment, the culture media may be supplemented with antibiotic-antimycotic, glutamax, penicillin/streptomycin, amphotericin B, TGF-β type I receptor inhibitor A83-01, nicotinamide, gastrin, non-essential amino acids, sodium pyruvate, R-Spondin, noggin, insulin growth factor 1 (IGF1), fibroblast growth factor 2 (FGF2), FGF10, epidermal growth factor (EGF), vascular endothelial growth factor (VEGF), Trombospondin (thrombospondin), ascorbic acid, Platelet-derived growth factor (PDGF), interleukin 6 (IE-6), IE-8, BMP4, BMP7, heparin, hydrocortisone, indomethacine, 3-Isobutyl-1-methylxanthine (IBMX), intralipids, bone morphogenetic protein 7 (BMP7), B27, A 83-01, N-Acetyl-L-cysteine, nicotinamide, Y-27632, RhoKinase inhibitor, primocin, ROCK inhibitor, or a combination thereof.

In one example embodiment, one or more organoid avatars disclosed herein may be cultured in a culture media for from about 1 day to about 60 days, such as from about 3 days to about 50 days, such as from about 7 days to about 45 days, such as from about 10 days to about 40 days, such as from about 14 days to about 30 days, or any range therebetween. For instance, in one example embodiment, one or more organoid avatars disclosed herein may be cultured in a culture media for from about 3 days to about 50 days. In another example embodiment, one or more organoid avatars disclosed herein may be cultured in a culture media for from about 7 days to about 45 days. In another example embodiment, one or more organoid avatars disclosed herein may be cultured in a culture media for from about 10 days to about 40 days. In another example embodiment, one or more organoid avatars disclosed herein may be cultured in a culture media for from about 14 days to about 30 days.

In one example embodiment, methods for screening a drug may include contacting the organoid avatars disclosed herein with a drug. In one example embodiment, a single drug may contact the organoid avatars. In another example embodiment, two or more drugs may contact the organoid avatars. For instance, the drug may be administered in vitro or ex vivo (e.g., by contacting the organoid with the drug) or, alternatively, in vivo (e.g., by administering the drug to a subject). As such, methods disclosed herein useful for treating a subject afflicted with a condition that would benefit from an increased immune response, such as an infection or a cancer.

In one example embodiment, the drug may be an agent useful for treating a cancer. The term “treating” as used herein refers to partially or completely alleviating, improving, relieving, inhibiting progression, and/or reducing incidence of one or more symptoms of a disease, disorder, and/or condition, e.g., cancer.

For instance, the cancer may include, but is not limited to, adrenal cortical cancer, advanced cancer, anal cancer, aplastic anemia, bile duct cancer, bladder cancer, bone cancer, bone metastasis, brain tumors, brain cancer, breast cancer, childhood cancer, cancer of unknown primary origin, Castleman disease, cervical cancer, colon cancer, colorectal cancer, central nervous system (CNS) cancer, endometrial cancer, esophagus cancer, Ewing family of tumors, eye cancer, gallbladder cancer, gastrointestinal carcinoid tumors, gastrointestinal stromal tumors, gestational trophoblastic disease, Hodgkin disease, Kaposi sarcoma, renal cell carcinoma, laryngeal and hypopharyngeal cancer, acute lymphocytic leukemia, acute myeloid leukemia, chronic lymphocytic leukemia, chronic myeloid leukemia, chronic myelomonocytic leukemia, liver cancer, non-small cell lung cancer (NSCLC), small cell lung cancer, lung carcinoid tumor, lymphoma of the skin, malignant mesothelioma, multiple myeloma, myelodysplastic syndrome, nasal cavity and paranasal sinus cancer, nasopharyngeal cancer, neuroblastoma, non-Hodgkin lymphoma, oral cavity and oropharyngeal cancer, osteosarcoma, ovarian cancer, pancreatic cancer, penile cancer, pituitary tumors, prostate cancer, retinoblastoma, rhabdomyosarcoma, salivary gland cancer, sarcoma in adult soft tissue, basal and squamous cell skin cancer, melanoma, small intestine cancer, stomach cancer, testicular cancer, throat cancer, thymus cancer, thyroid cancer, uterine sarcoma, vaginal cancer, vulvar cancer, and/or Waldenstrom macroglobulinemia. For instance, the cancer may include breast cancer, colon cancer, CNS, leukemia, melanoma, prostate, or renal cancer. In one example embodiment, the breast cancer may be triple negative breast cancer (TNBC), estrogen receptor-positive breast cancer, metastatic breast cancer, HER2 positive breast cancer, or a combination thereof. In one example embodiment, the cancer may be colon cancer. In another example embodiment, the cancer may be colorectal cancer.

In one example embodiment, the drug may be a therapeutic agent. In another example embodiment, the therapeutic agent may be an anticancer therapeutic. For instance, the anticancer therapeutic may be a tyrosine kinase inhibitor, a BRAF inhibitor, a Wnt inhibitor, or an EGFR inhibitor.

For instance, the anti-cancer therapeutic may include, but is not limited to, nutlin, bemaciclib, imatinib, gefitinib, erlotinib, sunitinib, lapatinib, nilotinib, sorafenib, temsirolimus, everolimus, pazopanib, crizotinib, ruxolitinib, vandetenib, axitinib, bosutinib, cabozantinib, ponatinib, regorafenib, ibrutinib, trametinib, perifosine, bortezomib, carfilzomib, marizomib batimastat, neovastat, prinomastat, rebimistat, ganetespib, epotoside, daunorubicin, cisplatin, dabrafenib, cabazitaxel, 5-flurouracil (5-FU), mercaptopurine, or a combination thereof. In one example embodiment, the anticancer therapeutic may be nutlin. In another example embodiment, the anticancer therapeutic may be ibrutinib. In another example embodiment, the anticancer therapeutic may be lapatinib. In another example embodiment, the anticancer therapeutic may be epotoside. In another example embodiment, the anticancer therapeutic may be daunorubicin. In another example embodiment, the anticancer therapeutic may be cisplatin. In another example embodiment, the anticancer therapeutic may be dabrafenib. In another example embodiment, the anticancer therapeutic may be cabazitaxel. In another example embodiment, the anticancer therapeutic may be 5-FU. In another example embodiment, the anticancer therapeutic may be mercaptopurine.

In one example embodiment, the anticancer therapeutic may be a Wnt inhibitor. For instance, the Wnt inhibitor may be LGK974, demethoxycurcumin, CCT036477, KY02111, WAY-316606, SFRP, IWP, C59, Ant1.4Br/Ant 1.4C1, ivermectin, niclosamide, sulforaphane, or a combination thereof. In one example embodiment, the Wnt inhibitor may be LGK974. In another example embodiment, the Wnt inhibitor may be C59. In another example embodiment, the Wnt inhibitor may be demethoxycurcumin.

In one example embodiment, monitoring the drug effect in the organoid avatars may include measuring resistance and/or sensitivity of an organoid avatar to the drug. An increased sensitivity or a reduced sensitivity to a therapeutic treatment may be measured using known method in the art including, but not limited to, cell proliferative assays and cell death assays.

In one example embodiment, the sensitivity of organoid avatars disclosed herein to a drug may be measured by a decrease of the population frequency of an organoid avatar from about 2% or more down to 0.02% or less in the presence of a drug, compared to the population frequency of an organoid avatar in the absence of said drug. In another example embodiment, the sensitivity of organoid avatars disclosed herein to a drug may be measured by a decrease of population frequency of an organoid avatar 0.5-fold down to 0.01-fold or more, compared to the same in the absence of said drug.

In one example embodiment, resistance of organoid avatars disclosed herein to a drug may be measured by an increase of copy numbers of an organoid avatar from about 20% or more, such as 30%, 40%, 50%, 60%, 70%, 80%, 90%, 95%, 97.5%, 98%, 99%, 99.9%, or more, compared to treatment resistance in the absence of said drug. In another example embodiment, resistance of organoid avatars disclosed herein to a drug may be measured by an increase of copy numbers of an organoid avatar from about 2-fold, 3-fold, 4-fold, 5-fold, 10-fold, 15-fold, 20-fold or more, compared to treatment resistance in the absence of said drug.

As used herein, “copy number” of a barcode nucleic acid refers to the number of DNA sequences in a cell (e.g., germline and/or somatic) encoding a particular gene product. Generally, for a given gene, a mammal has two copies of each gene. The copy number can be increased, however, by gene amplification or duplication, or reduced by deletion. For example, germline copy number changes include changes at one or more genomic loci, wherein said one or more genomic loci are not accounted for by the number of copies in the normal complement of germline copies in a control (e.g., the normal copy number in germline DNA for the same species as that from which the specific germline DNA and corresponding copy number were determined). Somatic copy number changes include changes at one or more genomic loci, wherein said one or more genomic loci are not accounted for by the number of copies in germline DNA of a control (e.g., copy number in germline DNA for the same subject as that from which the somatic DNA and corresponding copy number were determined). In one example embodiment, organoid copy number may be quantified relative to all the other organoids, and relative to the starting population of organoids. The absolute counts of each barcode may be proportional to the relative contributions of each organoid to the mixture.

In one example embodiment, evaluating the barcoded organoid avatar gene copy number in a sample may involve a Southern Blot. In a Southern Blot, the genomic DNA (typically fragmented and separated on an electrophoretic gel) is hybridized to a probe specific for the target region. Comparison of the intensity of the hybridization signal from the probe for the target region with the control probe signal from analysis of normal genomic DNA (e.g., a non-amplified portion of the same or related cell, tissue, organ, etc.) provides an estimate of the relative copy number of the target nucleic acid.

In one example embodiment, amplification-based assays may be used to measure copy numbers of a barcoded organoid avatar. In such amplification-based assays, the nucleic acid sequences act as a template in an amplification reaction (e.g., Polymerase Chain Reaction (PCR). In a quantitative amplification, the amount of amplification product will be proportional to the amount of template in the original sample. Comparison to appropriate controls, e.g., healthy tissue, provides a measure of the copy number.

Methods of “quantitative” amplification are well-known to those of skill in the art. For example, quantitative PCR involves simultaneously co-amplifying a known quantity of a control sequence using the same primers. This provides an internal standard that may be used to calibrate the PCR reaction. The known nucleic acid sequence for the genes is sufficient to enable one of skill in the art to routinely select primers to amplify any portion of the gene. Fluorogenic quantitative PCR may also be used in the methods of the present invention. In fluorogenic quantitative PCR, quantitation is based on the amount of fluorescence signals, e.g., TaqMan and SYBR green.

Other suitable amplification methods include, but are not limited to, ligase chain reaction (LCR), transcription amplification, self-sustained sequence replication, dot PCR, and linker adapter PCR, etc.

In one example embodiment, a barcoded organoid avatar may be sequenced using high-throughput sequencing, also known as next generation sequencing or deep sequencing. For instance, a nucleic acid target molecule (e.g., an organoid) labeled with a barcode may be sequenced with the barcode to produce a single read and/or contig containing the sequence, or portions thereof, of both the target molecule and the barcode.

In another example embodiment, a barcoded organoid avatar may be sequenced using high-throughput single-cell RNA-seq and/or targeted nucleic acid profiling (for example, sequencing, quantitative reverse transcription polymerase chain reaction, and the like), where the RNAs from different cells are tagged individually, allowing a single library to be created while retaining the cell identity of each read.

In one example embodiment, DNA may be purified from the mixture of organoids. PCR using primers that flank the barcodes and are common to all organoids may be used to generate a single PCR product that is a mixture of all barcodes present in the mixture. The relative contributions of each barcode in the PCR product may be determined by single-molecule sequencing and counting the number of each barcode read in the sequencing data.

Methods disclosed herein demonstrate the utility of patient-derived organoids (PDOs) barcoding combined with molecular detection to assess drug sensitivity in mixed PDO populations. In one example embodiment, methods may be useful for identifying drug sensitivity or resistance linked to known somatic mutations. The methods disclosed herein may be useful for making clinical choices for cancer patients by avoiding harsh chemotherapy regimens for which their tumors are resistant, and choosing treatments more likely to work based on positive results obtained from the organoid avatars. By integrating detailed genetic profiling with responsive organoid models, clinicians may better predict therapeutic outcomes and tailor treatments to exploit the unique vulnerabilities of cancer, thereby improving patient outcomes in a clinical setting.

The preceding description is exemplary in nature and is not intended to limit the scope, applicability or configuration of the disclosure in any way. Various changes to the described embodiments may be made in the function and arrangement of the elements described herein without departing from the scope of the disclosure.

Here and throughout the specification and claims, range limitations are combined and interchanged, such ranges are identified and include all the sub-ranges contained therein unless context or language indicates otherwise. For example, all ranges disclosed herein are inclusive of the endpoints, and the endpoints are independently combinable with each other.

This written description uses examples to disclose the present disclosure, including the best mode, and also to enable any person skilled in the art to practice the disclosure, including making and using any devices or systems and performing any incorporated methods. The patentable scope of the disclosure is defined by the claims, and may include other examples that occur to those skilled in the art. Such other examples are intended to be within the scope of the claims if they include structural elements that do not differ from the literal language of the claims, or if they include equivalent structural elements with insubstantial differences from the literal language of the claims.

It is to be understood that the terminology used herein is for the purpose of describing particular embodiments only and is not intended to limit the scope of the present disclosure. In this specification and in the claims that follow, reference will be made to a number of terms that shall be defined to have the following meanings.

Unless defined otherwise, all technical and scientific terms used herein have the same meaning as commonly understood by one of ordinary skill in the art to which this invention is related.

As used in this application and in the claims, the singular forms “a”, “an”, and “the” include the plural forms unless the context clearly dictates otherwise. Additionally, the term “includes” means “comprises”. The methods and compositions of the present disclosure, including components thereof, can comprise, consist of, or consist essentially of the essential elements and limitations of the embodiments described herein, as well as any additional or optional ingredients, components or limitations described herein or otherwise useful in biocidal compositions.

Unless otherwise indicated, all numbers expressing quantities of ingredients, properties such as molecular weight, percentages, and so forth, as used in the specification or claims are to be understood as being modified by the term “about”. Accordingly, unless otherwise indicated, implicitly or explicitly, the numerical parameters set forth are approximations that may depend on the desired properties sought and/or limits of detection under standard test conditions/methods. When directly and explicitly distinguishing embodiments from discussed prior art, the embodiment numbers are not approximate unless the word “about” is recited.

As used herein, “optional” or “optionally” means that the subsequently described material, event or circumstance may or may not be present or occur, and that the description includes instances where the material, event or circumstance is present or occurs and instances in which it does not. As used herein, “w/w %” and “wt %” mean by weight as relative to another component or a percentage of the total weight in the composition.

The term “about” is intended to mean approximately, in the region of, roughly, or around. When the term “about” is used in conjunction with a numerical range, it modifies that range by extending the boundaries above and below the numerical values set forth. Unless otherwise indicated, it should be understood that the numerical parameters set forth in the following specification and attached claims are approximations. At the very least, and not as an attempt to limit the application of the doctrine of equivalents to the scope of the claims, numerical parameters should be read in light of the number of reported significant digits and the application of ordinary rounding techniques.

The phrase “effective amount” means an amount of a compound that promotes, improves, stimulates, or encourages a response to the particular condition or disorder or the particular symptom of the condition or disorder.

Furthermore, certain aspects of the present disclosure may be better understood according to the following examples, which are intended to be non-limiting and exemplary in nature. Moreover, it will be understood that the compositions described in the examples may be substantially free of any substance not expressly described.

The present disclosure may be better understood with reference to the following examples.

### EXAMPLES

### Materials and Methods

### Organoid Isolation and Culture

Organoids were created from surgically resected colorectal cancers. Small pieces (1 cm square) of mucosal tissue were separated and washed in 1×PBS (Corning) supplemented with 0.1% Amphotericin B (Gibco) and 0.2% Primocin (Invivogen) to remove bacteria. Crypts were isolated with digestion and incubation in 1×PBS supplemented with 10% Collagenase A (Sigma) and 10 μM Y-27632 Rho Kinase inhibitor (Selleckchem) on a shaker at 37° C. for 40 min, followed by addition of 5% FBS (Gibco) and incubation for 5 minutes at room temp. Crypts were dislodged mechanically from tissue by pipetting up and down, separated into a new tube for additional wash, and centrifuged at 500×g for 5 min. The isolated stem cells were resuspended in 75 μL of ice-cold RGF BME Matrigel (R&D Systems) per well and plated in 12-well plates. The Matrigel was polymerized for 10-15 minutes at 37° C. humidity-controlled incubator with 5% CO₂ and subsequently covered with 2 mL per well of human colon organoids media (ENRA) containing 10 μM Y-27632 RhoKinase inhibitor (Selleckchem. The ENRA media contained Advanced DMEM/F-12 media (Gibco) supplemented with 10% R-Spondin, 10% Noggin (all CM produced in-house), 1×N2, 1×B27, 10 mM HEPES, 1× Glutamax, 1% Penicillin/Streptomycin, 0.25 ugmL-1 Amphotericin B, 50 ngmL-1 human Epidermal Growth Factor EGF (all from Gibco), 10 mM Nicotinamide, 1.25 mM N-acetyl-cysteine, 10 nM Gastrin (all from Sigma), 0.5 μM TGF-β type I receptor inhibitor A83-01 (Tocris Bioscience), 100 ugmL-1 Primocin (Invivogen). Media was changed every other day. For the first two changes of media after culturing, media contained 10 μM Y-27632 RhoKinase inhibitor (Selleckchem). Organoids were passaged 1:4 every 10-14 days. For passaging, organoids and Matrigel were covered with 2 ml/well of TrypLE (Gibco) plus 10 μM ROCK inhibitor (Selleckchem), dissociated by incubation at 37° C. for 5-7 min, then mechanically disrupted using a P1000 pipette and transferred into a 15-ml conical tube. Centrifuged at 500×g for 5 min. The pellet was resuspended and plated in ice-cold fresh Matrigel (R&D systems) and covered with ENRA media. Organoid lines were constantly tested for mycoplasma contamination and resulted negative.

### Cell Culture

All colon tumor organoids were grown and maintained in ENRA media containing 1% Penicillin/Streptomycin, 100 μg/ml Primocin, 0.25 ug/ml Amphotericin B. Organoids were kept in a 37° C. humidity-controlled incubator with 5% CO₂ and were maintained in exponential phase growth by passaging every 10-14 days and media was changed every other day. For viral transduction, 6 μg/ml polybrene was added. After viral transduction, 2 μg/ml puromycin were used for selection.

Organoid mixtures were treated with sub-lethal concentrations of each drug over a span of 28 days or 27 days with media changes and drug replenishment every other day, and the organoid pool replated in fresh Matrigel every week.

Isolation Individual Clone Barcode from the Barcoding Pool

ClonTracer Barcoding pool Library DNA was purchased from addgene (#67267). DNA was transformed into the bacteria and individual colonies were picked. Plasmids were purified from each individual clone and confirmed by sanger sequencing. Lentivirus of 72 individual unique barcodes were produced.

### Barcoding Patient's Derived Organoids—Viral Transduction

Prior to viral transduction, organoids were digested by incubation in TrypLE contained 10 μM ROCK inhibitor at 37° C. for 10-20 min. Then, cells were resuspended in 1× human colon tumor organoids media (ENRA) contained 10 μM ROCKi and Polybrene (6 μg/ml). Next, viral sufficient to achieve a multiplicity of infection (MOI) of approximately 3, were added to the cell mixture. The mixture was then transferred to a single well of a 24-well tissue culture plate, which was spun at 1000×g for 2 h at 30° C. After centrifugation, the plate was incubated at 37° C. incubator with 5% CO₂ for additional 3 h (total of 5 h). Following the incubation period, virus was washed out from the organoids with ice-cold PBS contained 10 μM ROCK. Transduced organoids then were embedded in ice-cold Matrigel and covered with ENRA media containing 10 μM ROCK inhibitor for the first two media changes (4 days). For all the other feedings, the organoids were maintained in ENRA media without ROCK. The Puromycin selection was started 2 days after the viral transduction and continued for 7 days.

### Next-Generation Sequencing (NGS) Assay

First, equal cell numbers of each barcoded organoids were mixed. Organoid mixtures were either plated in domes of Matrigel in tissue culture plates (48-wells, 12-wells or 6-well plates). A total of ˜250,000 cells per replicate were plated for drug treatment experiments. Organoid mixtures were treated with sub-lethal concentrations of each drug over a span of 28 or 27 days with media changes and drug replenishment every other day, and the organoid pool replated in fresh Matrigel every week. At weekly time points, 50% of the organoid pool was removed for sequencing and the remaining 50% was replated in fresh Matrigel. Throughout sampling for DNA purification, barcode PCR and amplicon sequencing, the size of each barcoded PDO population never dropped below a 1,000-fold bottleneck of 15,000 cells.

The genomic DNA of pooled barcoded PDOs were PCR amplified with primers that flank the DNA barcode sequences (Barcode_backbone_F and Barcode_backbone_R) and the products sequenced by Oxford Nanopore platform (LSK109 and NBD112.24 (Q20+)).

Barcode primers used for DNA barcode sequence amplification:

Barcode_backbone_forward primer, 5′-3′:
(SEQ ID NO: 1)
CGATTAGTGAACGGATCTCGAC
Barcode_backbone_reverse primer, 5′-3′:
(SEQ ID NO: 2)
CCATTTGTCTCGAGCTAGCATA

Primers used for ERBB3 amplification:

ERBB3_exon3_Forward primer, 5′-3′:
(SEQ ID NO: 18)
TTGCCCTGTTGTCTCTCTCA
ERBB3_exon3_Reverse primer, 5′-3′:
(SEQ ID NO: 19)
GTGGCTGGAGTTGGTGTTAT

### StarTrace PCR

Equal cell numbers of each barcoded organoids were mixed for pool-2. They were resuspended in a slurry of ENRA:Matrigel (3:4 ratio) and plated in rings around the rim of each well of a 96-well tissue culture plate (˜10,000 total cells per well). Select drugs from the approved Oncology Drug set VI were screened at maximum concentration of 10 μM and four serial dilutions over span of 10 days with refeeding every other day. After the treatment was completed, a multi-color fluorescent probe-based real time PCR assay (Taqman) was performed to quantify each barcoded organoids within the mixture. The primers targeted the barcode sequence location, and each probe was designed in a way to detect one unique barcode sequence which allowed us to quantify each unique barcoded organoids within a mixture and detect the effect of any drug concentration on each one separately. A probe targeting common backbone sequence was used to internally normalize. The assay used 4 μL of 5× PerfeCTa Multiplex qPCR ToughMix (Quantabio), 6 μL of triplex Primers and Probes mix, gDNA as template and water to adjust the reaction volume to 20 μL. PCR cycling conditions: 1 min at 95° C. for 1 cycle; 10 s at 95° C., 30 s at 62° C. for 4 cycles; 10 s at 95° C., 30 s at 59° C. for 4 cycles; 10 s at 95° C., 30 s at 56° C. for 4 cycles; association curve from 95° C. to 55° C.

Primers and probes used for qPCR assay:

| SEQ | | |
|---|---|---|
| ID | | |
| NO: | Name | Sequence |
| 01 | Barcode_forward primer, | CGATTAGTGAACGGATCTCGAC |
| | 5′-3′: | |
| 02 | Barcode_reverse primer, | CCATTTGTCTCGAGCTAGCATA |
| | 5′-3′: | |
| 03 | Barcode_backbone_probe: | /5TexRd-XN/ACTGACTGCAGTCTGAGTCTGACA/3IAbRQSp/ |
| 04 | P1-A9_probe: | /5HEX/AGTGACTGT/ZEN/GTGAGTGAGAGTGTCAGT/3IABkFQ/ |
| 05 | P1-B5_probe: | /5Cy5/TGACAGAGA/TAO/GTCTCTCACAGTGTGACA/3IAbRQSp/ |
| 06 | P1-B11_probe: | /56-FAM/ACTGTCTGT/ZEN/GACAGACTGAGTCAGTGA/3IABkFQ/ |
| 07 | P1-C6_probe: | /5Cy5/ACAGTGAGT/TAO/CAGAGAGTCTGAGACTGAG/3IAbRQSp/ |
| 08 | P1-C9_probe: | /5HEX/AGACTGTGA/ZEN/GTGTCAGACTCAGACTGT/3IABkFQ/ |
| 09 | P1-D1_probe: | /5Cy5/TGAGACTCT/TAO/CTCAGTCTCAGTCTCAGACA/3IAbRQSp/ |
| 10 | P1-G6_probe: | /56-FAM/TGTCACTCA/ZEN/GTGTGAGTCTGAGACTGT/3IABkFQ/ |
| 11 | P3-B12_probe: | /56-FAM/ACAGTCTGA/ZEN/CAGTGAGTGTGTGTCAC/3IABkFQ/ |
| 12 | P3-C6_probe: | /56-FAM/TCTGTGAGT/ZEN/CTGTGAGTGAGTGACACT/3IABkFQ/ |
| 13 | P3-D7_probe: | /5Cy5/AGTGTCTGA/TAO/CTGTCAGTGACAGAGTGT/3IAbRQSp/ |
| 14 | P3-E7_probe: | /5HEX/TGTCAGAGT/ZEN/CAGTCTGAGACAGTGACA/3IABkFQ/ |
| 15 | P3-E8_probe: | /56-FAM/ACAGTGTGT/ZEN/GACAGAGAGACTGAGT/3IABkFQ/ |
| 16 | P4-A2_probe: | /5HEX/AGTGACTGT/ZEN/CTGACTGACTCTGACTCAC/3IABkFQ/ |
| 17 | P4-A3_probe: | /5Cy5/AGTCTCTCA/TAO/GTGTGACAGTGTGTCTGA/3IAbRQSp/ |

The following 10× primer and probe mix was made for each pair: 5 μM forward primer; 5 μM reverse primer; and 2.5 μM probe.

A total of 5 different mixtures, each containing an equal amount of 4 different primers and probe, was prepared as follow:

| Mixtures | Barcode_FAM | Barcode_HEX | Barcode_Cy5 | Barcode_TEXAS RED |
|---|---|---|---|---|
| 1 | F147T_P1-G6 | F130T_P1-A9 | F158T_P4-A3 | Barcode_Backbone |
| 2 | F143T_P3-E8 | F186T_P4-A2 | F131T_P1-B5 | Barcode_Backbone |
| 3 | F108T_P3-B12 | F182_P3-E7 | F197T_P3-D7 | Barcode_Backbone |
| 4 | F133T_P3-C6 | F172T_P1-C1 | F109T_P1-C6 | Barcode_Backbone |
| 5 | F169T_P1-B11 | F179T_P1-C9 | P071316T_P1-D1 | Barcode_Backbone |

### NGS Analysis

Raw FASTQ files were analyzed by a fuzzy text matching program (UGREP). Example of the command:
ugrep -Z3 -c ‘guide_forward_seq|guide_reverse-complement_seq’ sample_fastq_file.fastq
-Z3 is the fuzzy option to allow 3 mismatches.

Each FASTQ file from a single sample replicate was queried for all expected barcodes and the resulting raw counts normalized to the sum of all counts. Log fold changes relative to the no-drug control was used to assess drug-dependent effects on Darwinian fitness.

### StarTrace Analysis

For every well, the difference between the Ct signal of each BC PDO and the TexasRed Ct (Backbone) signal was first calculated. Next, the log fold change (LFC) of each BC PDO's frequency in drug-treated samples was calculated, normalized to DMSO. A negative value means fewer total cells in the drug-treated samples compared to DMSO.

### Example 1

### High-Throughput Barcode Deconvolution Using Next-Generation Sequencing

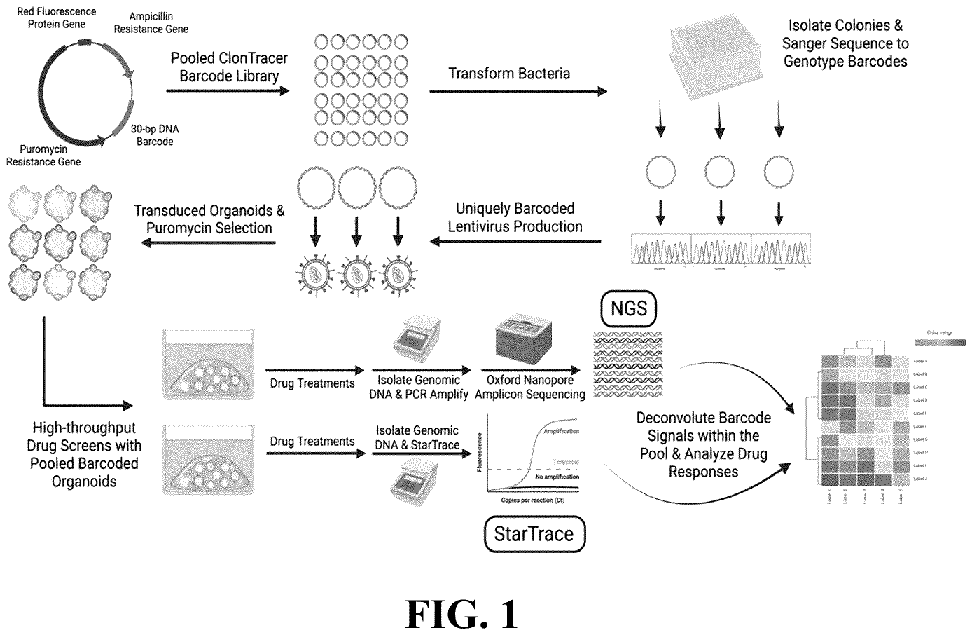

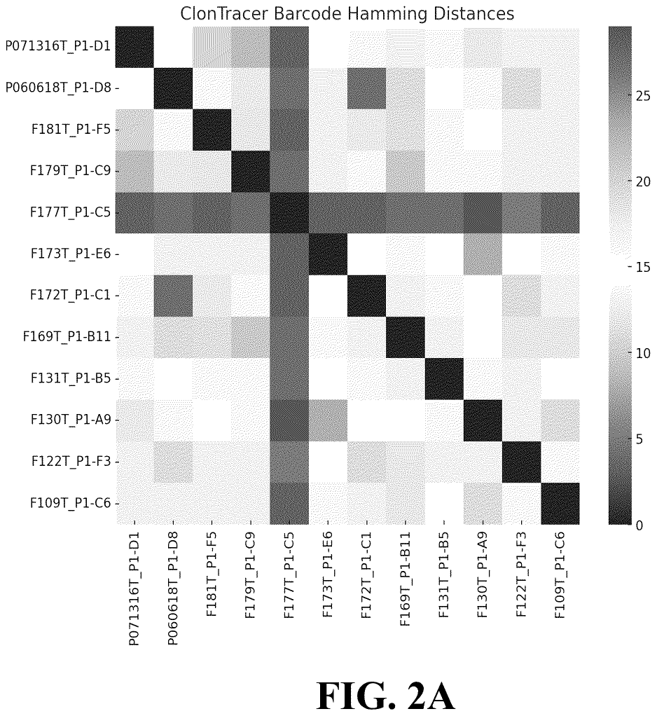

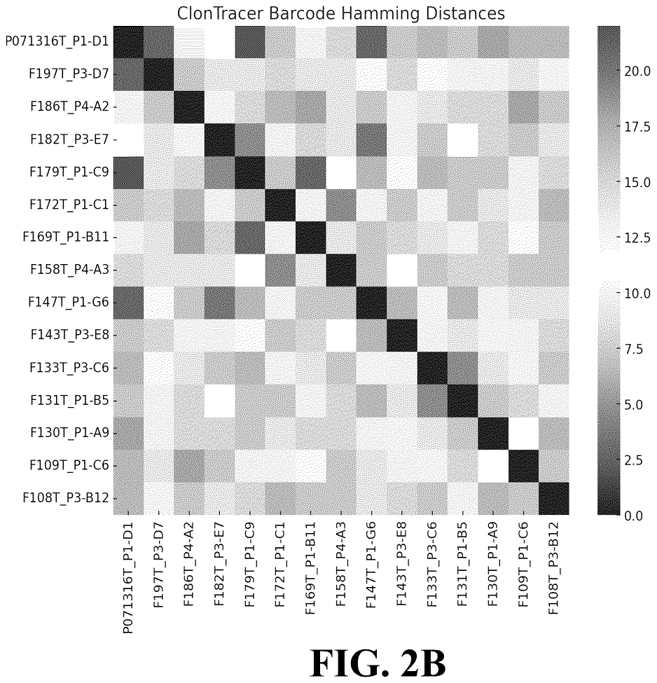

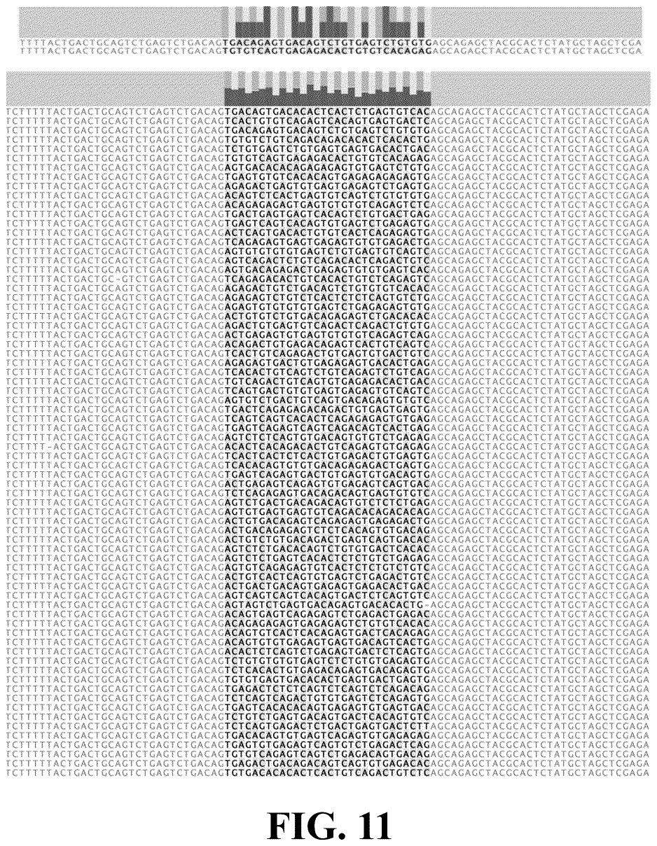

Molecular barcoding, involving the integration of short, non-coding DNA sequences into the genomes of cell populations via viral transduction has been used previously to monitor the invisible sub-clones within the diverse tumor cell populations, providing insights into tumor development and progression. Herein, an NGS-based method has been developed that enables to track each patient-derived organoids (PDOs) present in a mixture in response to treatment with various cancer drugs. This method uses oxford nanopore amplicon sequencing to track the frequency of each unique barcode during extended culturing of the mixture in various drug conditions. Seventy-two (72) randomly picked barcode clones were isolated from a high-complexity ClonTracer barcoding library (addgene #67267) and generated infectious viral particles for tagging individual organoids (FIG. 1). Each barcode, comprising a 30-base pair unique DNA sequence, was identified by Sanger sequencing (FIG. 11). Barcode sequences were selected that sufficiently divergent from one another (FIGS. 2A-2B), to allow reliable identification of each barcode when mixtures are sequences by nanopore-platform amplicon sequencing. All organoids were characterized by tumor/normal whole exome sequencing and select cancer-causing somatic mutations are shown in Table 1.

TABLE 1
Relationship between drug response and select somatic mutations.

| Organoid | Nutlin | Ibrutinib | Lapatinib | TKI | BRAF | ERBB3 | KRAS | TP53 |
|---|---|---|---|---|---|---|---|---|
| F108T | R | | | | | | | p.Ser215Asn |
| F109T | R | R | R | R | p.Val600Glu | | | p.Arg234Cys |
| F122T | R | | | | | | | p.Asn92fs |
| F133T | R | | | | | | | p.Arg273His |
| F143T | R | | | | | | | |
| F169T | R | ? | R | R | | | p.Gly12Asp | p.Gly245Ser |
| F172T | R | | | | | | | p.Arg248Gln |
| F179T | R | | | | | | | p.Arg136His |
| F181T | R | R | R | R | | | p.Gly12Asp | p.Ser127_Gln128dup |
| F182T | R | | | | | | | p.Cys176Tyr |
| F197T | R | | | | | | | p.Arg248Trp |
| P060618T | R | S | R | ? | | | p.Gly12Ala | p.Arg248Trp |
| P071316T | R | S | S | S | | p.Ser1119Cys | | p.Arg248Gln |
| F130T | S | R | R | R | | | p.Gly12Val | |
| F131T | S | ? | R | R | | | p.Gly13Asp | |
| F147T | S | | | | p.Val600Glu | | | p.Val34fs |
| F158T | S | | | | | | | |
| F173T | S | S | ? | S | | p.Val104Leu | | |
| F177T | S | | | | | | | |
| F186T | S | | | | | | | |

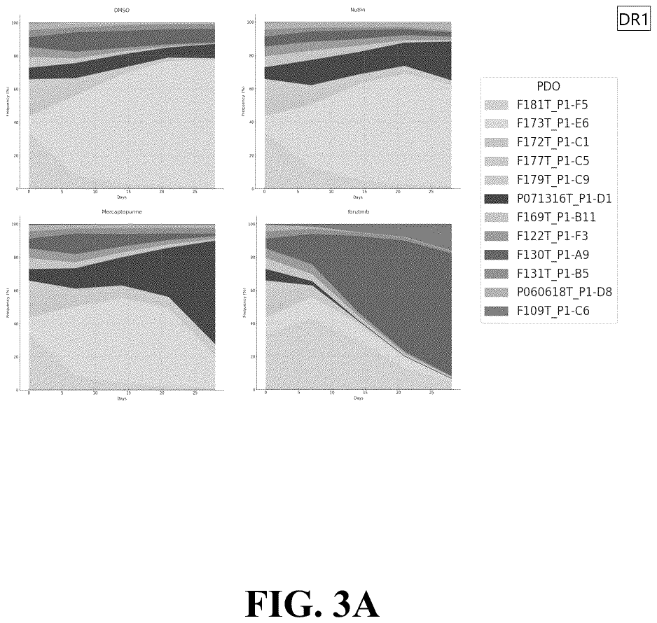

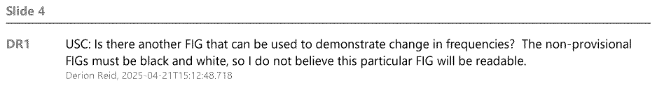

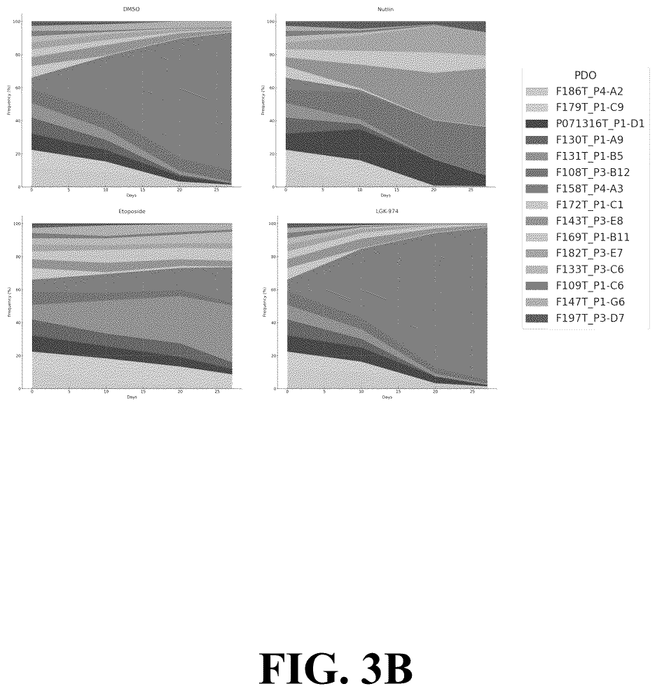

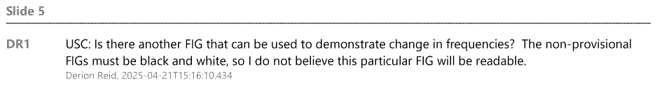

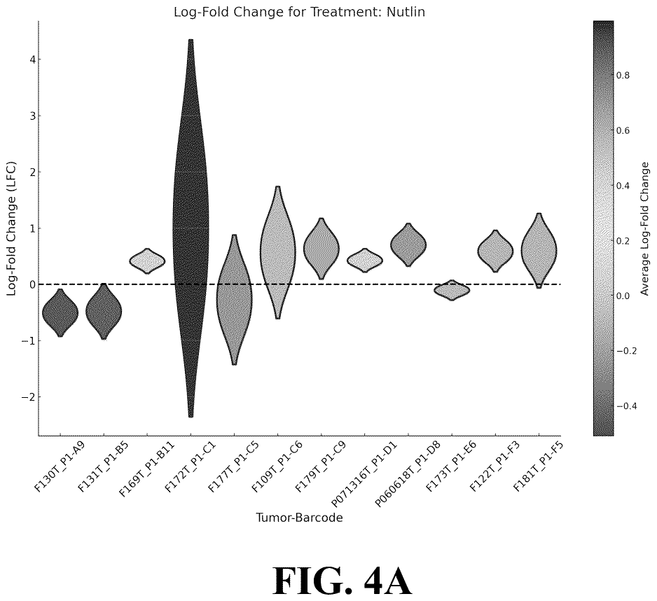

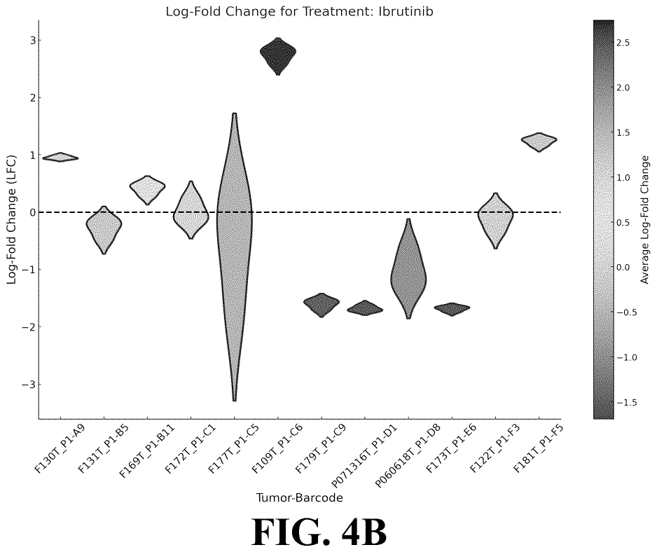

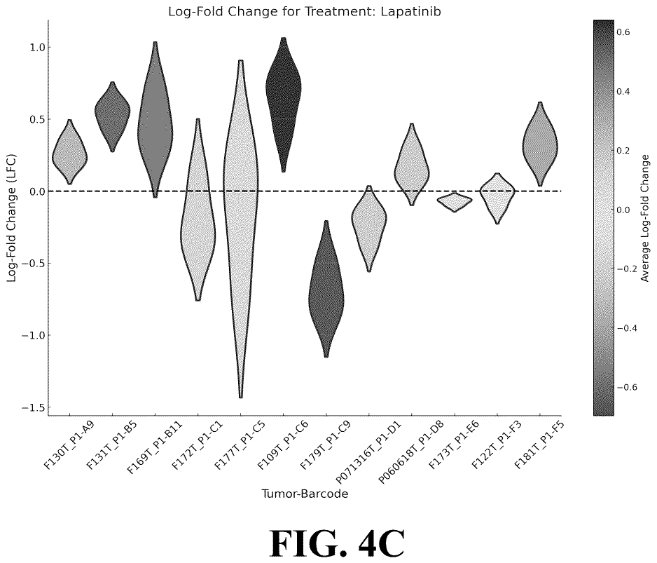

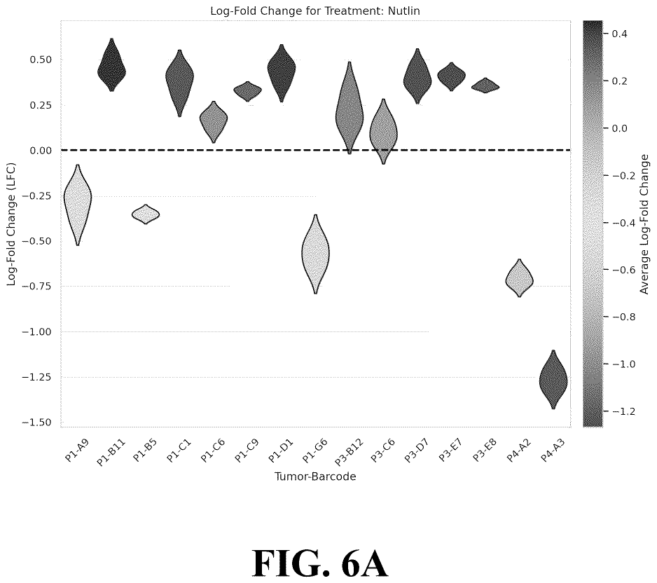

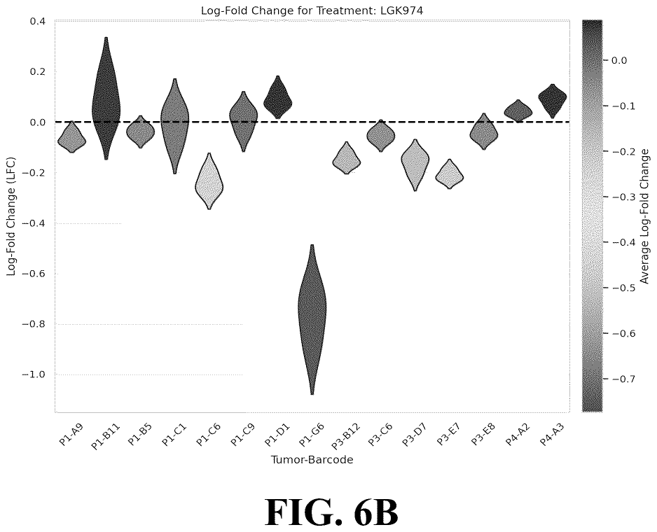

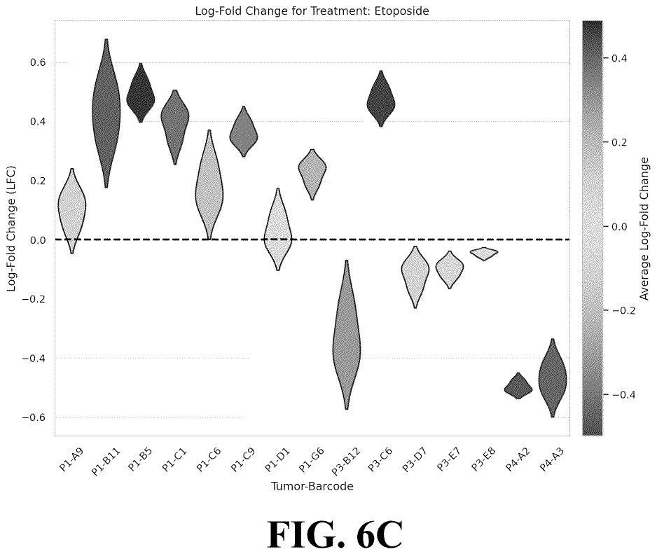

Each individual PDO was transduced with a unique lentivirus clone containing a single DNA barcode and selected with puromycin. Each barcoded PDO contains lentiviral insertions containing a unique 30-bp barcode sequence flanked by lentiviral backbone sequences common to all barcoded organoids. These barcoded PDOs can be combined into bespoke organoid pools for different applications. Two different custom pools were created to test cancer drug sensitivities. Pool-1 contained 12 barcoded PDOs and was treated with 5 different chemotherapy drugs, including nutlin-3a (MDM2 inhibitor), 5-Fluorouracil (5-FU), Mercaptopurine, Lapatinib, and Ibrutinib. Treatments were done with sub-lethal concentrations of each drug over a span of 28 days, with weekly sampling. Pool-2 contained 15 barcoded PDOs and was treated with nutlin-3a, LGK-974 (PORCN inhibitor), and Etoposide (TOPO isomerases inhibitor). Each sampling involved removing 50% of the organoid pool for qPCR and amplicon sequencing, while the remaining 50% was replated in fresh Matrigel for continued drug treatment. Throughout the process of DNA isolation, PCR, and sequencing, the size of each barcoded PDO population never dropped below a 1,000-fold bottleneck of 15,000 cells. Genomic DNA prepared from the time point samples of the pools was used for amplicon sequencing of the barcode locus on the Oxford Nanopore platform. The FASTQ files were analyzed using fuzzy grep commands (UGREP) and enumerated the counts of all expected barcodes. The change in frequencies of each barcode was computed over time to compute relative Darwinian finesses of each condition compared to the no drug control (FIGS. 3A-3B). As expected, nutlin resistance associated with TP53 mutations, indicating the presence of both TP53-Wildtype (TP53-WT) and TP53-Mutant (TP53-MT) organoids. Overall, 13 out of 20 PDOs were resistant to nutlin (FIGS. 3A-3B, 4A, and 6A), with 12 of these 13 nutlin-resistant PDOs found by whole exome sequencing (WES) to harbor inactivating somatic mutations in the TP53 gene (Table 1). One nutlin-resistant PDO, F143T_P3-E8 had no detectable TP53 somatic mutation. This nutlin resistance may result from either an undetected TP53 mutation or MDM2 amplification (Table 1; FIGS. 4A and 6A).

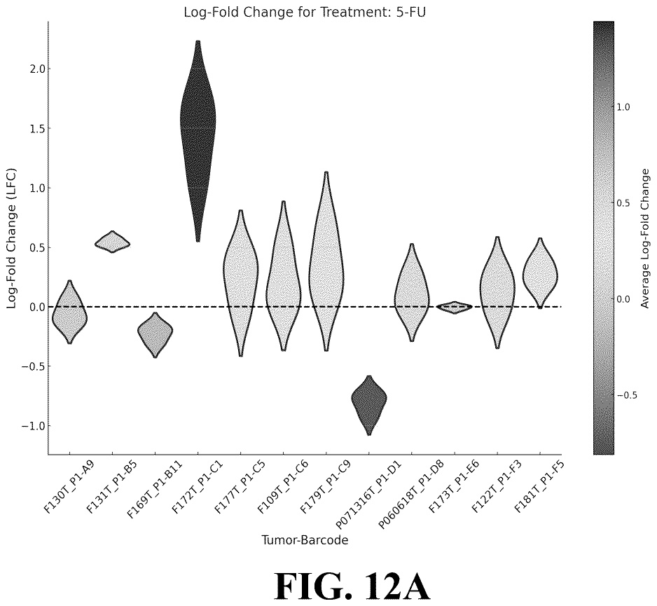

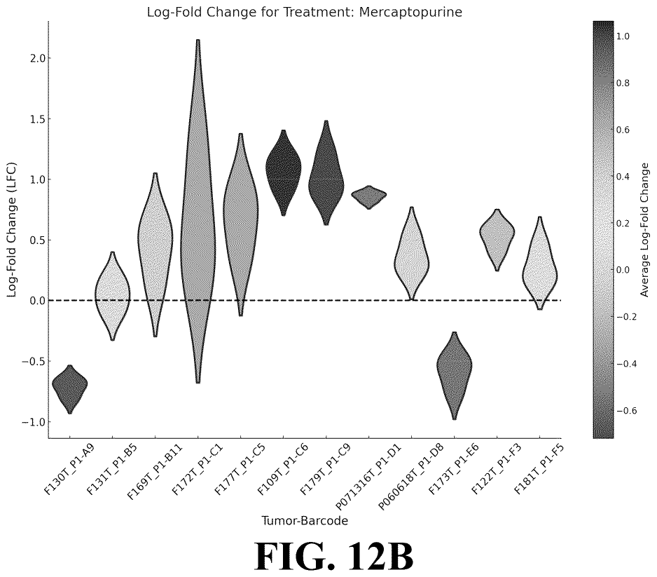

Six organoids demonstrated resistance to tyrosine kinase inhibitors (TKIs), Ibrutinib and Lapatinib, and this correlated with the presence of BRAF and KRAS mutations. Two organoids (F173T-P1-E6 and P071316T_P1-D1) showed sensitivity to these inhibitors, which may be related to the presence of ERBB3 mutations in these tumors. (Table 1; FIGS. 4B-4C and 12A-12B).

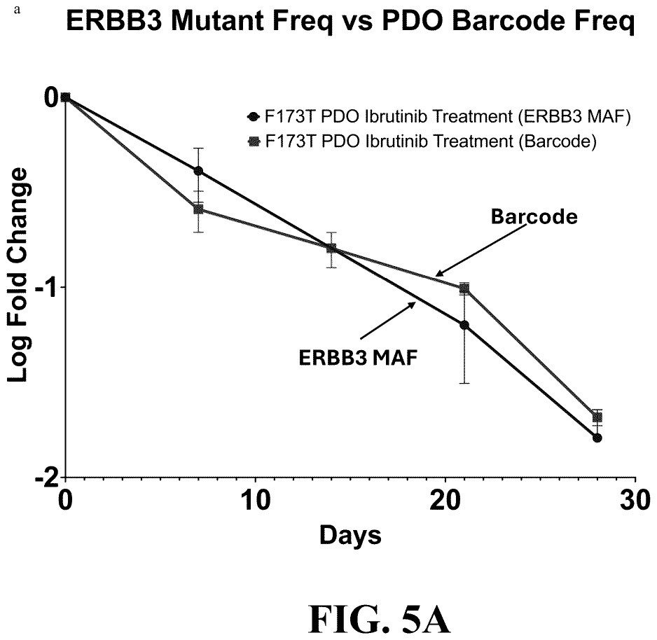

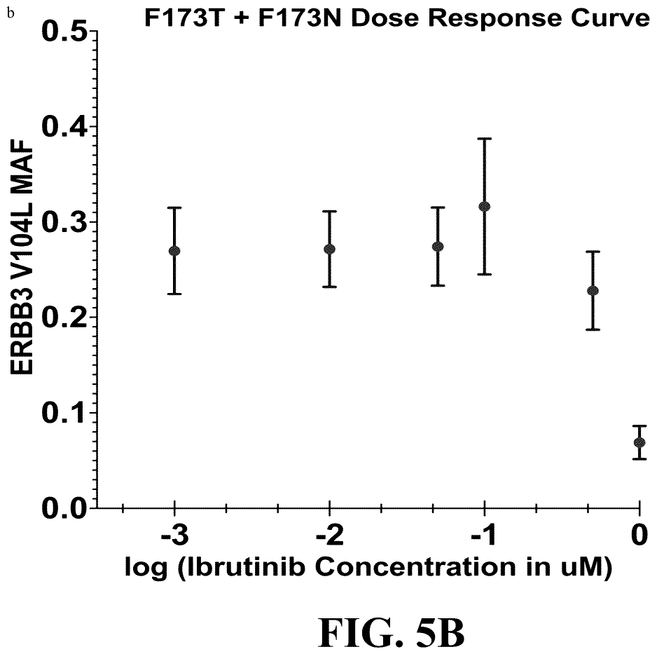

Ibrutinib is a tyrosine kinase inhibitor used to target Bruton's tyrosine kinase (BTK) in hematological malignancies. The F173T PDO exhibited sensitivity to Ibrutinib, despite lacking any detectable BTK mutation. However, F173T carries a somatic mutation to ERBB3 (Val104Leu) which may heterodimerize with and trans activate other receptor tyrosine kinases. FIG. 5A shows that the loss of mutant ERBB3 allele frequency with Ibrutinib treatment correlates well with the loss of the barcode for F173T. To determine if Ibrutinib sensitivity is a somatically acquired phenotype, we performed a drug response experiment using an unbarcoded mixture of F173T and patient-matched normal, F173N (FIG. 5B), followed by PCR sequencing of the mutant ERBB3 amplicon. The mutant ERBB3 allele was significantly depleted at high Ibrutinib concentrations. This result aligns with studies showing Ibrutinib inhibits ERBB receptor phosphorylation, leading to the suppression of key survival pathways such as PI3K/AKT and MAPK/ERK in solid tumors. Overall, this result demonstrates the utility of functional testing in organoids to identify unexpected, yet relevant drug sensitivities.

Topoisomerase inhibitors are sometimes used in colon cancer treatment; however, outcomes remain difficult to predict due to the lack of reliable biomarkers of clinical response. PDOs showing both etoposide sensitivity and resistance are clearly identifiable (FIGS. 3B and 6C). Phenotyping cancers ex vivo may have practical utility for patients deciding on topoisomerase inhibitor therapy.

Previously, it was discovered through a genome-wide CRISPR KO library screen that the F147T PDO had a PORCN gene knockout dependency, consistent with a role for self-stimulating (autocrine) WNT pathway activation in this RNF43-mutant tumor. The present study utilized LGK-974, a drug that targets the Wnt-specific acyltransferase porcupine (PORCN) and blocks maturation of endogenous WNT ligand. F147T_P1-G6 grown without external Wnt supplementation, demonstrated significant sensitivity to the PORCN inhibitor. This indicates that this tumor has the capability to activate the Wnt pathway using its own WNT ligand, thereby operating through an autocrine rather than paracrine mechanism. The data here shows the ability of this platform to detect the sensitivity of one RNF43 mutant colon tumor to LGK-974 within the pool using NGS.

### Example 2

StarTrace, a Barcode qPCR Quantification Method

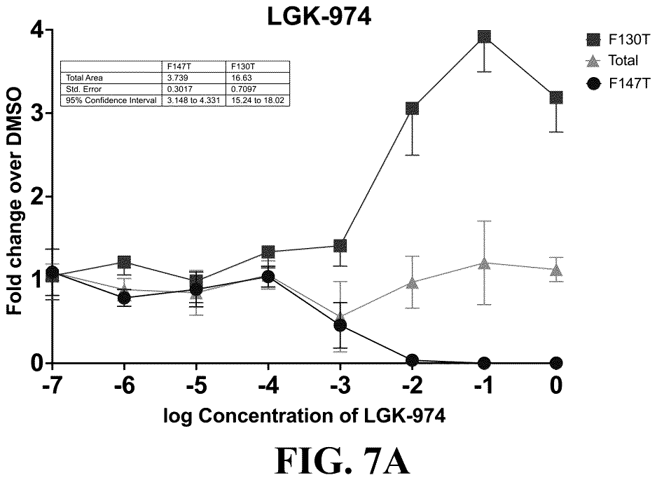

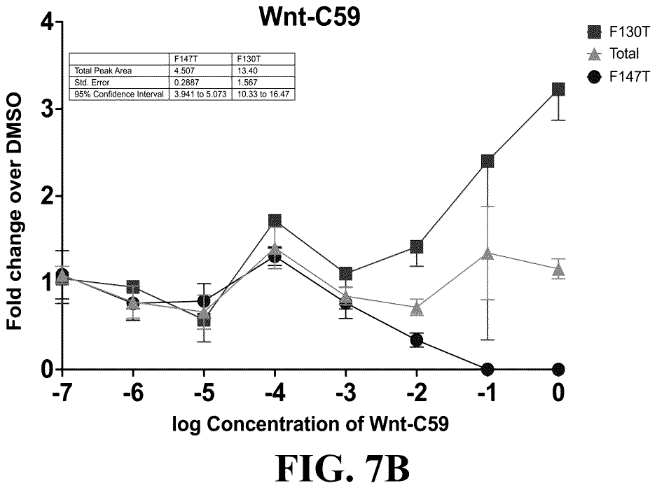

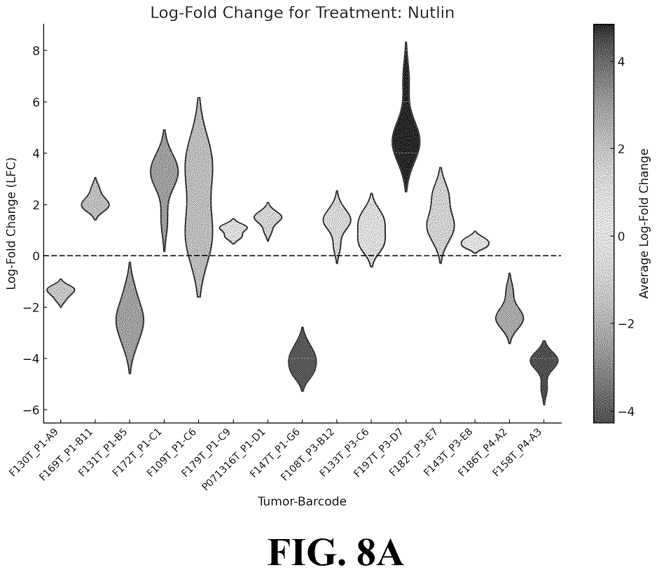

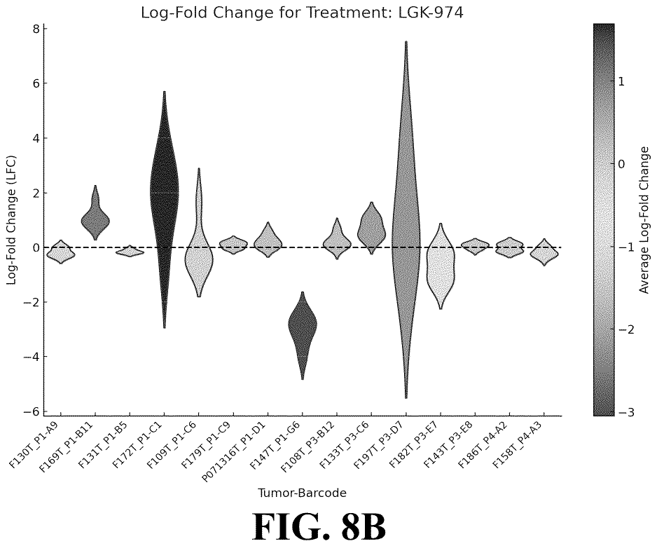

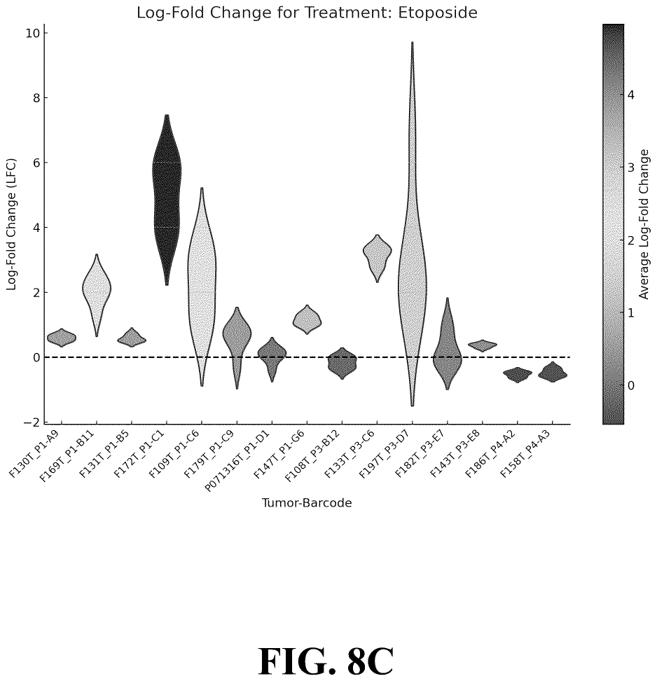

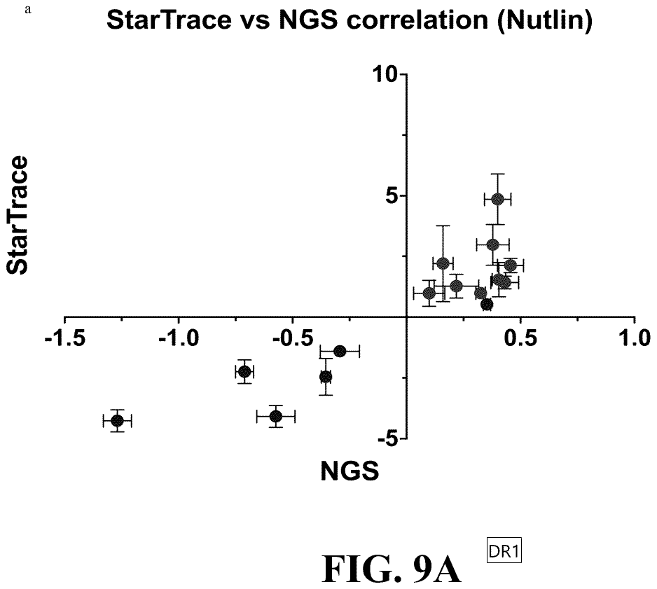

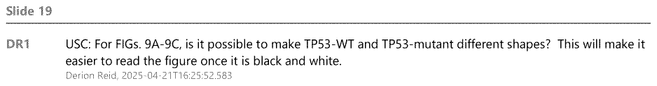

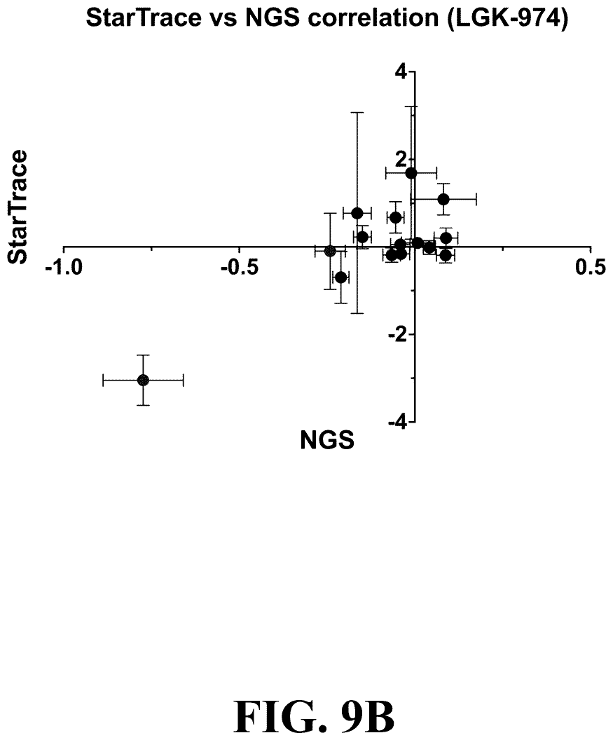

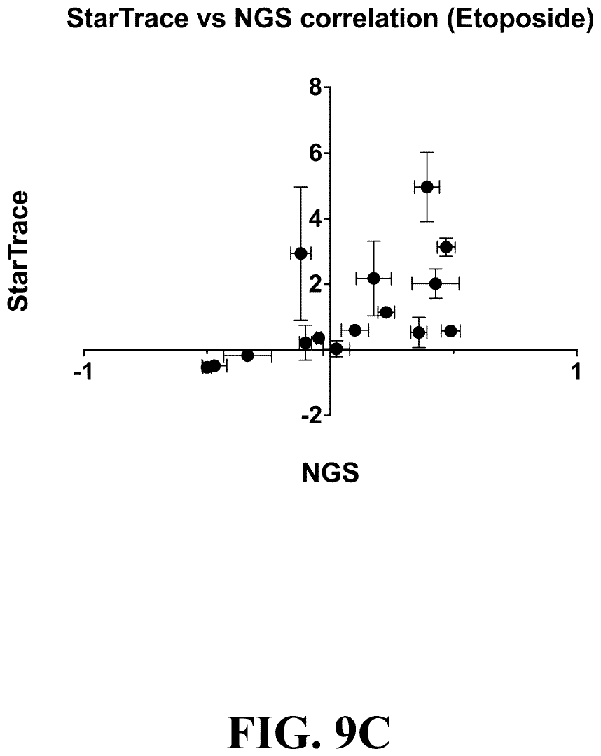

To augment NGS quantification of individual barcodes, a rapid and cost-effective qPCR approach was developed for quantifying barcodes within a mixture. A multi-color qPCR was utilized with common forward and reverse amplification primers and unique TaqMan probes targeting individual DNA barcodes. This allowed the rapid quantification of each barcoded PDO within the mixture and detect drug dependent effects on Darwinian fitness. To demonstrate the principal, a 50/50 mixture of two BC PDOs, (F147T_P1-G6 and F130T_P1-A9) was first created and treated the mixture with eight different concentrations of the PORCN inhibitors, LGK-974 and Wnt-C59. After the treatment was completed, genomic DNA was prepared and used StarTrace PCR to detect the two unique barcode sequences in the mixture and determine the quantity of each organoid in each condition. FIGS. 7A-7B show the relative sensitivities of F147T (harboring a somatic mutation to RNF43) and F130T_P1-A9. Next, the StarTrace PCR assay was applied to a pool of samples (Pool-2) treated with four different drugs. The results shown in FIGS. 8A-8C, support the drug response pattern observed by NGS (FIGS. 6A-6C). These findings demonstrate a robust correlation between NGS and StarTrace PCR method (FIGS. 9A-9C), demonstrating the reliability of StarTrace primer/probe sets in accurately quantifying barcode signals in complex mixtures.

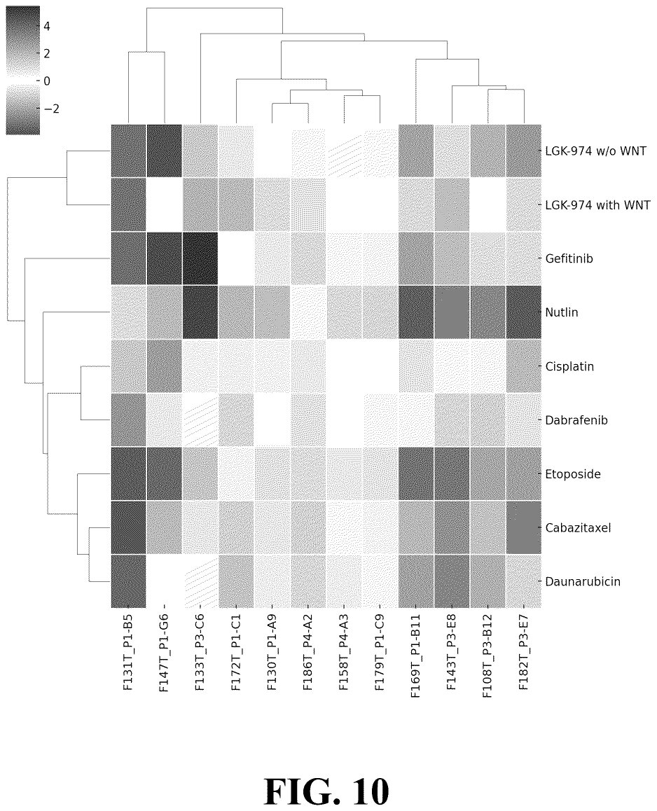

Subsequently, the StarTrace assay was miniaturized using Pool-2 barcoded PDOs. To do so, a simple high-throughput organoid plating method in a 96-well plate format was applied and treated them with four different concentrations of 8 different drugs. In this miniaturized platform, 12 of the 15 organoids in all time points of the untreated samples were successfully detected. Two organoids fell below the limit of qPCR detection. The heatmap in FIG. 10 shows the relative sensitivities of 12 out of 15 BC PDOs. The expected sensitivity to PORCN inhibitor was observed in the RNF43 mutated tumor (F147T_P1-G6). Interestingly, when this tumor was grown in the presence of external Wnt supplement, the efficacy of the PORCN inhibitor was notably diminished, aligning with the mechanism of PORCIN inhibitor action. StarTrace PCR correctly detected the expected nutlin sensitivity in the TP53-WT tumors F130T_P1-A9, F131_P1-B5, F147T_P1-G6, F186T_P4-A2, and F158T_P1-A3. These organoids' nutlin sensitivities were independently confirmed through nutlin sensitivity assays on individual tumors grown separately and the results aligned with TP53 mutations obtained from nanopore-based TP53 amplicon sequencing and by Illumina-based whole exome sequencing. Sensitivity to Gefitinib, Etoposide, Daunorubicin, and Cisplatin were detected in select individual organoids, reflecting the diverse landscapes of therapeutic vulnerabilities within the sampled tumor populations. These findings underscore the potential for tailored therapeutics approaches based on specific drug sensitivity profiles observed in the organoids, highlighting the advantages of using this rapid, high-throughput, and precision assay.

These and other modifications and variations to the present invention may be practiced by those of ordinary skill in the art, without departing from the spirit and scope of the present invention, which is more particularly set forth in the appended claims. In addition, it should be understood that aspects of the various embodiments may be interchanged both in whole or in part. Furthermore, those of ordinary skill in the art will appreciate that the foregoing description is by way of example only and is not intended to limit the invention so further described in such appended claims.

What is claimed:

1. A method for simultaneous monitoring the relative sensitivities of different organoids to a therapeutic for treating a cancer, the method comprising:
a) obtaining a sample comprising a plurality of barcoded organoid avatars;
b) contacting the sample with a therapeutic;
c) detecting in the sample at a first point in time the copy number, amount, and/or activity of one or more organoids;
d) repeating step c) at a subsequent point in time; and
e) comparing the copy number of one or more organoids detected in steps c) and d) to monitor the progression of the effectiveness of the therapeutic.

2. The method of claim 1, wherein the organoid is derived from a group consisting of colorectal cells, intestinal cells, crypt cells, rectal cells, lung cells, liver cells, breast cells, skin cells, pancreatic cells, endocrine cells, exocrine cells, ductal cells, renal cells, adrenal cells, thyroid cells, pituitary cells, parathyroid cells, prostate cells, stomach cells, oesophageal cells, and ovary cells.

3. The method of claim 2, wherein the organoid is derived from colorectal cells.

4. The method of claim 1, wherein an increase in copy numbers of an organoid avatar from about 25% or more signals sensitivity of the barcoded organoid avatars to the therapeutic.

5. The method of claim 1, wherein an increase in copy numbers of an organoid avatar of about 2-fold or more signals sensitivity of the barcoded organoid avatars to the therapeutic.

6. The method of claim 1, wherein a decrease in copy numbers of an organoid avatar from about 25% or more signals resistance of the barcoded organoid avatars to the therapeutic.

7. The method of claim 1, wherein a decrease in copy numbers of an organoid avatar of about 2-fold or more signals resistance of the barcoded organoid avatars to the therapeutic.

8. The method of claim 1, wherein the first and/or at least one subsequent sample comprises ex vivo or in vitro samples.

9. The method of claim 1, wherein the barcoded organoid avatars comprise a nucleic acid barcode.

10. The method of claim 9, wherein the nucleic acid barcode has a sequence length of from about 6 base pairs (bp) to about 50 bp.

11. The method of claim 1, wherein the first and/or the at least one subsequent sample is a portion of a single sample or a pooled samples.

12. The method of claim 1, further comprising contacting the sample with two or more immunotherapies.

13. The method of claim 1, wherein the therapeutic agent is a tyrosine kinase inhibitor, a BRAF inhibitor, a Wnt inhibitor, an EGFR inhibitor, or a combination thereof.

14. The method of claim 1, wherein the therapeutic is selected from a group consisting of nutlin, bemaciclib, imatinib, gefitinib, erlotinib, sunitinib, lapatinib, nilotinib, sorafenib, temsirolimus, everolimus, pazopanib, crizotinib, ruxolitinib, vandetenib, axitinib, bosutinib, cabozantinib, ponatinib, regorafenib, ibrutinib, trametinib, perifosine, bortezomib, carfilzomib, marizomib, batimastat, neovastat, prinomastat, rebimistat, ganetespib, epotoside, daunorubicin, cisplatin, dabrafenib, cabazitaxel, 5-flurouracil (5-FU), mercaptopurine, LGK974, demethoxycurcumin, CCTO36477, KY02111, WAY-316606, SFRP, IWP, C59, Ant1.4Br/Ant 1.4C1, ivermectin, niclosamide, and sulforaphane.

15. The method of claim 14, wherein the therapeutic is nutlin, ibrutinib, lapatinib, bemaciclib, imatinib, epotoside, daunorubicin, cisplatin, dabrafenib, cabazitaxel, 5-flurouracil (5-FU), mercaptopurine, or a combination thereof.

16. The method of claim 14, wherein the therapeutic is LGK974, C59, or a combination thereof.

17. The method of claim 1, wherein the cancer is selected from a group consisting of adrenal cortical cancer, advanced cancer, anal cancer, aplastic anemia, bile duct cancer, bladder cancer, bone cancer, bone metastasis, brain tumors, brain cancer, breast cancer, childhood cancer, cancer of unknown primary origin, Castleman disease, cervical cancer, colon cancer, colorectal cancer, central nervous system (CNS) cancer, endometrial cancer, esophagus cancer, Ewing family of tumors, eye cancer, gallbladder cancer, gastrointestinal carcinoid tumors, gastrointestinal stromal tumors, gestational trophoblastic disease, Hodgkin disease, Kaposi sarcoma, renal cell carcinoma, laryngeal and hypopharyngeal cancer, acute lymphocytic leukemia, acute myeloid leukemia, chronic lymphocytic leukemia, chronic myeloid leukemia, chronic myelomonocytic leukemia, liver cancer, non-small cell lung cancer (NSCLC), small cell lung cancer, lung carcinoid tumor, lymphoma of the skin, malignant mesothelioma, multiple myeloma, myelodysplastic syndrome, nasal cavity and paranasal sinus cancer, nasopharyngeal cancer, neuroblastoma, non-Hodgkin lymphoma, oral cavity and oropharyngeal cancer, osteosarcoma, ovarian cancer, pancreatic cancer, penile cancer, pituitary tumors, prostate cancer, retinoblastoma, rhabdomyosarcoma, salivary gland cancer, sarcoma in adult soft tissue, basal and squamous cell skin cancer, melanoma, small intestine cancer, stomach cancer, testicular cancer, throat cancer, thymus cancer, thyroid cancer, uterine sarcoma, vaginal cancer, vulvar cancer, and/or Waldenstrom macroglobulinemia.

18. The method of claim 17, wherein the cancer is colorectal cancer.

19. The method of claim 1, wherein the barcoded organoid avatar is detected using polymerase chain reaction (PCR), next-generation sequencing, or a hybridization assay.

20. The method of claim 1, wherein the barcoded organoid avatar is detected using polymerase chain reaction (PCR) and next-generation sequencing.


SEQUENCE LISTING (93 sequences)

SEQ ID NO: 1 - DNA, 22 bp, synthetic construct
  source 1..22: mol_type = other DNA
cgattagtgaacggatctcgac

SEQ ID NO: 2 - DNA, 22 bp, synthetic construct
  source 1..22: mol_type = other DNA
ccatttgtctcgagctagcata

SEQ ID NO: 3 - DNA, 24 bp, synthetic construct
  source 1..24: mol_type = other DNA
actgactgcagtctgagtctgaca

SEQ ID NO: 4 - DNA, 18 bp, synthetic construct
  source 1..18: mol_type = other DNA
gtgagtgagagtgtcagt

SEQ ID NO: 5 - DNA, 18 bp, synthetic construct
  source 1..18: mol_type = other DNA
gtctctcacagtgtgaca

SEQ ID NO: 6 - DNA, 18 bp, synthetic construct
  source 1..18: mol_type = other DNA
gacagactgagtcagtga

SEQ ID NO: 7 - DNA, 19 bp, synthetic construct
  source 1..19: mol_type = other DNA
cagagagtctgagactgag

SEQ ID NO: 8 - DNA, 18 bp, synthetic construct
  source 1..18: mol_type = other DNA
gtgtcagactcagactgt

SEQ ID NO: 9 - DNA, 20 bp, synthetic construct
  source 1..20: mol_type = other DNA
ctcagtctcagtctcagaca

SEQ ID NO: 10 - DNA, 18 bp, synthetic construct
  source 1..18: mol_type = other DNA
gtgtgagtctgagactgt

SEQ ID NO: 11 - DNA, 17 bp, synthetic construct
  source 1..17: mol_type = other DNA
cagtgagtgtgtgtcac

SEQ ID NO: 12 - DNA, 18 bp, synthetic construct
  source 1..18: mol_type = other DNA
ctgtgagtgagtgacact

SEQ ID NO: 13 - DNA, 18 bp, synthetic construct
  source 1..18: mol_type = other DNA
ctgtcagtgacagagtgt

SEQ ID NO: 14 - DNA, 18 bp, synthetic construct
  source 1..18: mol_type = other DNA
cagtctgagacagtgaca

SEQ ID NO: 15 - DNA, 16 bp, synthetic construct
  source 1..16: mol_type = other DNA
gacagagagactgagt

SEQ ID NO: 16 - DNA, 19 bp, synthetic construct
  source 1..19: mol_type = other DNA
ctgactgactctgactcac

SEQ ID NO: 17 - DNA, 18 bp, synthetic construct
  source 1..18: mol_type = other DNA
gtgtgacagtgtgtctga

SEQ ID NO: 18 - DNA, 20 bp, synthetic construct
  source 1..20: mol_type = other DNA
ttgccctgttgtctctctca

SEQ ID NO: 19 - DNA, 20 bp, synthetic construct
  source 1..20: mol_type = other DNA
gtggctggagttggtgttat

SEQ ID NO: 20 - DNA, 89 bp, synthetic construct
  source 1..89: mol_type = other DNA
ttttactgactgcagtctgagtctgacagtgacagagtgacagtctgtgagtctgtgtgagcagagctacgcactctatgctagctcga

SEQ ID NO: 21 - DNA, 89 bp, synthetic construct
  source 1..89: mol_type = other DNA
ttttactgactgcagtctgagtctgacagtgtgtcagtgagagacactgtgtcacagagagcagagctacgcactctatgctagctcga

SEQ ID NO: 22 - DNA, 94 bp, synthetic construct
  source 1..94: mol_type = other DNA
tctttttactgactgcagtctgagtctgacagtgacagtgacacactcactctgagtgtcacagcagagctacgcactctatgctagctcgaga

SEQ ID NO: 23 - DNA, 94 bp, synthetic construct
  source 1..94: mol_type = other DNA
tctttttactgactgcagtctgagtctgacagtcactgtgtcagagtcacagtgagtgactcagcagagctacgcactctatgctagctcgaga

SEQ ID NO: 24 - DNA, 94 bp, synthetic construct
  source 1..94: mol_type = other DNA
tctttttactgactgcagtctgagtctgacagtgacagagtgacagtctgtgagtctgtgtgagcagagctacgcactctatgctagctcgaga

SEQ ID NO: 25 - DNA, 94 bp, synthetic construct
  source 1..94: mol_type = other DNA
tctttttactgactgcagtctgagtctgacagtgtgtctgtcagacagacacactcacactgagcagagctacgcactctatgctagctcgaga

SEQ ID NO: 26 - DNA, 94 bp, synthetic construct
  source 1..94: mol_type = other DNA
tctttttactgactgcagtctgagtctgacagtctgtgagtctgtgagtgagtgacactgacagcagagctacgcactctatgctagctcgaga

SEQ ID NO: 27 - DNA, 94 bp, synthetic construct
  source 1..94: mol_type = other DNA
tctttttactgactgcagtctgagtctgacagtgtgtcagtgagagacactgtgtcacagagagcagagctacgcactctatgctagctcgaga

SEQ ID NO: 28 - DNA, 94 bp, synthetic construct
  source 1..94: mol_type = other DNA
tctttttactgactgcagtctgagtctgacagagtgacacacagagagagtgtgagtctgtgagcagagctacgcactctatgctagctcgaga

SEQ ID NO: 29 - DNA, 94 bp, synthetic construct
  source 1..94: mol_type = other DNA
tctttttactgactgcagtctgagtctgacagtgagtgtgtcacacagtgagagagagagtgagcagagctacgcactctatgctagctcgaga

SEQ ID NO: 30 - DNA, 94 bp, synthetic construct
  source 1..94: mol_type = other DNA
tctttttactgactgcagtctgagtctgacagagagactgagtgtgagtgagagtctgagtgagcagagctacgcactctatgctagctcgaga

SEQ ID NO: 31 - DNA, 94 bp, synthetic construct
  source 1..94: mol_type = other DNA
tctttttactgactgcagtctgagtctgacagacagtctcactgagtgtcagtctgtgtgtgagcagagctacgcactctatgctagctcgaga

SEQ ID NO: 32 - DNA, 94 bp, synthetic construct
  source 1..94: mol_type = other DNA
tctttttactgactgcagtctgagtctgacagacagagagagtgagtgtgtgtcagagtctcagcagagctacgcactctatgctagctcgaga

SEQ ID NO: 33 - DNA, 94 bp, synthetic construct
  source 1..94: mol_type = other DNA
tctttttactgactgcagtctgagtctgacagtgactgagtgagtcacagtctgtgactgagagcagagctacgcactctatgctagctcgaga

SEQ ID NO: 34 - DNA, 94 bp, synthetic construct
  source 1..94: mol_type = other DNA
tctttttactgactgcagtctgagtctgacagtgagtcagtcacagtgtgagtctgagagtgagcagagctacgcactctatgctagctcgaga

SEQ ID NO: 35 - DNA, 94 bp, synthetic construct
  source 1..94: mol_type = other DNA
tctttttactgactgcagtctgagtctgacagactcagtgacactgtgtcactcagagagtgagcagagctacgcactctatgctagctcgaga

SEQ ID NO: 36 - DNA, 94 bp, synthetic construct
  source 1..94: mol_type = other DNA
tctttttactgactgcagtctgagtctgacagtcagagagtgagtgagagtgtgtgagactgagcagagctacgcactctatgctagctcgaga

SEQ ID NO: 37 - DNA, 94 bp, synthetic construct
  source 1..94: mol_type = other DNA
tctttttactgactgcagtctgagtctgacagagtgtgtgtgtgagtctgtgagtgtcagtcagcagagctacgcactctatgctagctcgaga

SEQ ID NO: 38 - DNA, 94 bp, synthetic construct
  source 1..94: mol_type = other DNA
tctttttactgactgcagtctgagtctgacagagtcagactctgtcagacactcagactgtcagcagagctacgcactctatgctagctcgaga

SEQ ID NO: 39 - DNA, 94 bp, synthetic construct
  source 1..94: mol_type = other DNA
tctttttactgactgcagtctgagtctgacagagtgacagagactgagagtgtgtgagtcacagcagagctacgcactctatgctagctcgaga

SEQ ID NO: 40 - DNA, 93 bp, synthetic construct
  source 1..93: mol_type = other DNA
tctttttactgactgcgtctgagtctgacagtcagagacactgtcacactgtctcagagtcagcagagctacgcactctatgctagctcgaga

SEQ ID NO: 41 - DNA, 94 bp, synthetic construct
  source 1..94: mol_type = other DNA
tctttttactgactgcagtctgagtctgacagagagactgtctgacagtctgtgtgtcacacagcagagctacgcactctatgctagctcgaga

SEQ ID NO: 42 - DNA, 94 bp, synthetic construct
  source 1..94: mol_type = other DNA
tctttttactgactgcagtctgagtctgacagagagagtctgtctcactctctcagtgtctgagcagagctacgcactctatgctagctcgaga

SEQ ID NO: 43 - DNA, 94 bp, synthetic construct
  source 1..94: mol_type = other DNA
tctttttactgactgcagtctgagtctgacagagagtgtgtgtgtgagtctgagagagtgtgagcagagctacgcactctatgctagctcgaga

SEQ ID NO: 44 - DNA, 94 bp, synthetic construct
  source 1..94: mol_type = other DNA
tctttttactgactgcagtctgagtctgacagactgtgtctgtgacagagagtctgacacacagcagagctacgcactctatgctagctcgaga

SEQ ID NO: 45 - DNA, 94 bp, synthetic construct
  source 1..94: mol_type = other DNA
tctttttactgactgcagtctgagtctgacagagactgtgagtgtcagactcagactgtgtgagcagagctacgcactctatgctagctcgaga

SEQ ID NO: 46 - DNA, 94 bp, synthetic construct
  source 1..94: mol_type = other DNA
tctttttactgactgcagtctgagtctgacagactgagagtgtgagtgtgtgtcagagtcagagcagagctacgcactctatgctagctcgaga

SEQ ID NO: 47 - DNA, 94 bp, synthetic construct
  source 1..94: mol_type = other DNA
tctttttactgactgcagtctgagtctgacagacagactgtgagacagagtcactgtcagtcagcagagctacgcactctatgctagctcgaga

SEQ ID NO: 48 - DNA, 94 bp, synthetic construct
  source 1..94: mol_type = other DNA
tctttttactgactgcagtctgagtctgacagtcactgtcagagactgtgagtgtgactgtcagcagagctacgcactctatgctagctcgaga

SEQ ID NO: 49 - DNA, 94 bp, synthetic construct
  source 1..94: mol_type = other DNA
tctttttactgactgcagtctgagtctgacagagagagtgactgtgagagagtgacactgagagcagagctacgcactctatgctagctcgaga

SEQ ID NO: 50 - DNA, 94 bp, synthetic construct
  source 1..94: mol_type = other DNA
tctttttactgactgcagtctgagtctgacagtcacactgtcagtctgtcagagtctgtcagagcagagctacgcactctatgctagctcgaga

SEQ ID NO: 51 - DNA, 94 bp, synthetic construct
  source 1..94: mol_type = other DNA
tctttttactgactgcagtctgagtctgacagtgtcagactgtcagtgtgagagacactgacagcagagctacgcactctatgctagctcgaga

SEQ ID NO: 52 - DNA, 94 bp, synthetic construct
  source 1..94: mol_type = other DNA
tctttttactgactgcagtctgagtctgacagtcagtgactgtgtgagtgagagtgtcagtcagcagagctacgcactctatgctagctcgaga

SEQ ID NO: 53 - DNA, 94 bp, synthetic construct
  source 1..94: mol_type = other DNA
tctttttactgactgcagtctgagtctgacagagtgtctgactgtcagtgacagagtgtgtcagcagagctacgcactctatgctagctcgaga

SEQ ID NO: 54 - DNA, 94 bp, synthetic construct
  source 1..94: mol_type = other DNA
tctttttactgactgcagtctgagtctgacagtgactcagagagacagactgtgagtgagtgagcagagctacgcactctatgctagctcgaga

SEQ ID NO: 55 - DNA, 94 bp, synthetic construct
  source 1..94: mol_type = other DNA
tctttttactgactgcagtctgagtctgacagtcagtcagtcacactcagagagagtgtgagagcagagctacgcactctatgctagctcgaga

SEQ ID NO: 56 - DNA, 94 bp, synthetic construct
  source 1..94: mol_type = other DNA
tctttttactgactgcagtctgagtctgacagtgagtcagagtcagtcagacagtcactgagagcagagctacgcactctatgctagctcgaga

SEQ ID NO: 57 - DNA, 94 bp, synthetic construct
  source 1..94: mol_type = other DNA
tctttttactgactgcagtctgagtctgacagagtctctcagtgtgacagtgtgtctgagagagcagagctacgcactctatgctagctcgaga

SEQ ID NO: 58 - DNA, 93 bp, synthetic construct
  source 1..93: mol_type = other DNA
tcttttactgactgcagtctgagtctgacagacactcacagacactgtcagagtgtgagagagcagagctacgcactctatgctagctcgaga

SEQ ID NO: 59 - DNA, 94 bp, synthetic construct
  source 1..94: mol_type = other DNA
tctttttactgactgcagtctgagtctgacagtcactcactctcactgtgagactgtgagtgagcagagctacgcactctatgctagctcgaga

SEQ ID NO: 60 - DNA, 94 bp, synthetic construct
  source 1..94: mol_type = other DNA
tctttttactgactgcagtctgagtctgacagtcacacagtgtgtgacagagagactgagtgagcagagctacgcactctatgctagctcgaga

SEQ ID NO: 61 - DNA, 94 bp, synthetic construct
  source 1..94: mol_type = other DNA
tctttttactgactgcagtctgagtctgacagtgagtcagagtgactgtgagtgtgacagtgagcagagctacgcactctatgctagctcgaga

SEQ ID NO: 62 - DNA, 94 bp, synthetic construct
  source 1..94: mol_type = other DNA
tctttttactgactgcagtctgagtctgacagactgagagtcagagtgtgagagtcagtgacagcagagctacgcactctatgctagctcgaga

SEQ ID NO: 63 - DNA, 94 bp, synthetic construct
  source 1..94: mol_type = other DNA
tctttttactgactgcagtctgagtctgacagtctcagagagtgacagacagtgagtgtgtcagcagagctacgcactctatgctagctcgaga

SEQ ID NO: 64 - DNA, 94 bp, synthetic construct
  source 1..94: mol_type = other DNA
tctttttactgactgcagtctgagtctgacagagtctgactgacagacagtgtctctctgagagcagagctacgcactctatgctagctcgaga

SEQ ID NO: 65 - DNA, 94 bp, synthetic construct
  source 1..94: mol_type = other DNA
tctttttactgactgcagtctgagtctgacagagtgtgagtgagtgtcagacacagacacagagcagagctacgcactctatgctagctcgaga

SEQ ID NO: 66 - DNA, 94 bp, synthetic construct
  source 1..94: mol_type = other DNA
tctttttactgactgcagtctgagtctgacagactgtgacagagtcagagagtgagagactgagcagagctacgcactctatgctagctcgaga

SEQ ID NO: 67 - DNA, 94 bp, synthetic construct
  source 1..94: mol_type = other DNA
tctttttactgactgcagtctgagtctgacagactgacagagagtctctcacagtgtgacagagcagagctacgcactctatgctagctcgaga

SEQ ID NO: 68 - DNA, 94 bp, synthetic construct
  source 1..94: mol_type = other DNA
tctttttactgactgcagtctgagtctgacagactgtctgtgacagactgagtcagtgacacagcagagctacgcactctatgctagctcgaga

SEQ ID NO: 69 - DNA, 94 bp, synthetic construct
  source 1..94: mol_type = other DNA
tctttttactgactgcagtctgagtctgacagagtctctgacacagtctgtgtgactcacacagcagagctacgcactctatgctagctcgaga

SEQ ID NO: 70 - DNA, 94 bp, synthetic construct
  source 1..94: mol_type = other DNA
tctttttactgactgcagtctgagtctgacagagtctctgagtcacactctctgtctgagagagcagagctacgcactctatgctagctcgaga

SEQ ID NO: 71 - DNA, 94 bp, synthetic construct
  source 1..94: mol_type = other DNA
tctttttactgactgcagtctgagtctgacagagtgtcagagagtgtcactctctgtctgtcagcagagctacgcactctatgctagctcgaga

SEQ ID NO: 72 - DNA, 94 bp, synthetic construct
  source 1..94: mol_type = other DNA
tctttttactgactgcagtctgagtctgacagactgtcactcagtgtgagtctgagactgtcagcagagctacgcactctatgctagctcgaga

SEQ ID NO: 73 - DNA, 94 bp, synthetic construct
  source 1..94: mol_type = other DNA
tctttttactgactgcagtctgagtctgacagactgactgacagtgagagtgagacactgacagcagagctacgcactctatgctagctcgaga

SEQ ID NO: 74 - DNA, 94 bp, synthetic construct
  source 1..94: mol_type = other DNA
tctttttactgactgcagtctgagtctgacagagtcagtcagtcacagtgactctcagtgtcagcagagctacgcactctatgctagctcgaga

SEQ ID NO: 75 - DNA, 93 bp, synthetic construct
  source 1..93: mol_type = other DNA
tctttttactgactgcagtctgagtctgacagagtagtctgagtgacagagtgacacactgagcagagctacgcactctatgctagctcgaga

SEQ ID NO: 76 - DNA, 94 bp, synthetic construct
  source 1..94: mol_type = other DNA
tctttttactgactgcagtctgagtctgacagacagtgagtcagagagtctgagactgagacagcagagctacgcactctatgctagctcgaga

SEQ ID NO: 77 - DNA, 94 bp, synthetic construct
  source 1..94: mol_type = other DNA
tctttttactgactgcagtctgagtctgacagacagagagagtgagagagtctgtgtcacacagcagagctacgcactctatgctagctcgaga

SEQ ID NO: 78 - DNA, 94 bp, synthetic construct
  source 1..94: mol_type = other DNA
tctttttactgactgcagtctgagtctgacagacagtgtcactcacagagtgactcacagagagcagagctacgcactctatgctagctcgaga

SEQ ID NO: 79 - DNA, 94 bp, synthetic construct
  source 1..94: mol_type = other DNA
tctttttactgactgcagtctgagtctgacagacagtgtgtgagagtgagtgacagtcactgagcagagctacgcactctatgctagctcgaga

SEQ ID NO: 80 - DNA, 94 bp, synthetic construct
  source 1..94: mol_type = other DNA
tctttttactgactgcagtctgagtctgacagacactctcagtctgagagtgactctcagtgagcagagctacgcactctatgctagctcgaga

SEQ ID NO: 81 - DNA, 94 bp, synthetic construct
  source 1..94: mol_type = other DNA
tctttttactgactgcagtctgagtctgacagtctgtgtgtgtgagtctctgtgtgagagtgagcagagctacgcactctatgctagctcgaga

SEQ ID NO: 82 - DNA, 94 bp, synthetic construct
  source 1..94: mol_type = other DNA
tctttttactgactgcagtctgagtctgacagtctcacactgtgagacagagtgacagagtgagcagagctacgcactctatgctagctcgaga

SEQ ID NO: 83 - DNA, 94 bp, synthetic construct
  source 1..94: mol_type = other DNA
tctttttactgactgcagtctgagtctgacagtgtgtgagtgacacactgagtgactgagtgagcagagctacgcactctatgctagctcgaga

SEQ ID NO: 84 - DNA, 94 bp, synthetic construct
  source 1..94: mol_type = other DNA
tctttttactgactgcagtctgagtctgacagtgagactctctcagtctcagtctcagacagagcagagctacgcactctatgctagctcgaga

SEQ ID NO: 85 - DNA, 94 bp, synthetic construct
  source 1..94: mol_type = other DNA
tctttttactgactgcagtctgagtctgacagtctcagtcagactgtgtgagtctcagagtgagcagagctacgcactctatgctagctcgaga

SEQ ID NO: 86 - DNA, 94 bp, synthetic construct
  source 1..94: mol_type = other DNA
tctttttactgactgcagtctgagtctgacagtgagtcacacacagtgagagtgtgagtgacagcagagctacgcactctatgctagctcgaga

SEQ ID NO: 87 - DNA, 94 bp, synthetic construct
  source 1..94: mol_type = other DNA
tctttttactgactgcagtctgagtctgacagtctgtctgagtgacagtgactcacagtgtcagcagagctacgcactctatgctagctcgaga

SEQ ID NO: 88 - DNA, 94 bp, synthetic construct
  source 1..94: mol_type = other DNA
tctttttactgactgcagtctgagtctgacagtctcagtgagactctgactgagtgactcttagcagagctacgcactctatgctagctcgaga

SEQ ID NO: 89 - DNA, 94 bp, synthetic construct
  source 1..94: mol_type = other DNA
tctttttactgactgcagtctgagtctgacagtgacacagtgtgagtcagagtgtgagagagagcagagctacgcactctatgctagctcgaga

SEQ ID NO: 90 - DNA, 94 bp, synthetic construct
  source 1..94: mol_type = other DNA
tctttttactgactgcagtctgagtctgacagtgagtgtgagagtcagtgtctgagactcagagcagagctacgcactctatgctagctcgaga

SEQ ID NO: 91 - DNA, 94 bp, synthetic construct
  source 1..94: mol_type = other DNA
tctttttactgactgcagtctgagtctgacagtgtgtcagagtcagtctgagacagtgacagagcagagctacgcactctatgctagctcgaga

SEQ ID NO: 92 - DNA, 94 bp, synthetic construct
  source 1..94: mol_type = other DNA
tctttttactgactgcagtctgagtctgacagtgagactgacagacagtcagtgtgagacacagcagagctacgcactctatgctagctcgaga

SEQ ID NO: 93 - DNA, 94 bp, synthetic construct
  source 1..94: mol_type = other DNA
tctttttactgactgcagtctgagtctgacagtgtgacacacactcactgtcagactgtctcagcagagctacgcactctatgctagctcgaga